(19) United States
(12) Patent Application Publication
(10) Pub. No.: US 2024/0148648 A1
(43) Pub. Date: May 9, 2024

(54) SUSTAINED RELEASE THERMOSETTING GELS COMPRISING ANESTHETIC DRUGS AND THE METHODS OF MAKING THE SAME

(71) Applicant: Pacira Therapeutics, Inc., Tampa, FL (US)

(72) Inventors: Scott Kelley, Burlington, MA (US); Ujjwal Joshi, Burlington, MA (US); Ami Jo, Burlington, MA (US); John Derek Jackson, Lexington, MA (US); Rebecca Senter, Boston, MA (US)

(21) Appl. No.: 18/549,536

(22) PCT Filed: Mar. 9, 2022
(86) PCT No.: PCT/US2022/019589

§ 371 Date: Sep. 7, 2023

Related U.S. Application Data
(60) Provisional application No. 63/159,229, filed on Mar. 10, 2021.

(51) Int. Cl. A61K 9/06; A61K 31/167; A61K 31/445; A61K 47/10; A61K 47/18; A61K 47/34

(52) U.S. Cl. CPC A61K 9/06; A61K 31/167; A61K 31/445; A61K 47/10; A61K 47/18; A61K 47/34

(57) ABSTRACT

The present disclosure provides compositions comprising a thermosensitive hydrogel formulation of anesthetic drugs and a triblock copolymer component, and methods of use and manufacture of the compositions for management of acute pain or pain associated with surgical procedure.

### RELATED APPLICATIONS

This application claims the benefit of U.S. Provisional Application No. 63/159,229, filed on Mar. 10, 2021, the contents of which is hereby incorporated by reference in their entirety.

### FIELD OF THE INVENTION

The present disclosure describes compositions and methods directed to treating or managing pain where the compositions comprise anesthetic drug of amino amide group formulated in one or more triblock co-polymers. The present disclosure also provides methods of manufacturing the compositions, as described herein.

### BACKGROUND OF THE INVENTION

There are an estimated 70 million surgical procedures completed in the United States each year (Owings 1998). Over 80% of patients who undergo surgical procedures experience acute postsurgical pain and ˜75% of those patients report their pain severity as moderate, severe or extreme (Chou 2016). In more than 50% of cases, patients report not receiving adequate pain relief following their procedure (Chou 2016). Inadequately controlled postsurgical pain can have both short- and long-term effects, including negative effects on quality of life, function and functional recovery, the risk of postsurgical complications, and the risk of persistent postsurgical pain.

There is a substantial unmet need in postsurgical pain management for a non-opioid sensory blockade with an extended regional analgesia effect that does not materially compromise motor function. Orthopedic surgery generates the second-highest rates of opioid prescriptions among adults, with many patients using opioids both before and after surgery (Smith 2018), facilitating a path to potential addiction. A treatment that provides extended pain relief while not materially impairing motor function could reduce the need for opioids, allow for earlier ambulation and physical therapy, and provide the ability for patients to leave the hospital sooner after surgery.

Bupivacaine, lidocaine, and ropivacaine are a few of the common anesthetics used in surgery for post-surgical pain management. There is an unmet need for a pharmaceutical composition to deliver drugs including the above-mentioned anesthetics for durable pain relief with a single injection. Disclosed herein are compositions based on a thermosensitive hydrogel-based formulation developed for the delivery of active pharmaceutical ingredients (APIs), like bupivacaine, lidocaine, and ropivacaine for the purpose of post-surgical pain management. The present invention is exemplified by using the anesthetics bupivacaine, lidocaine, and ropivacaine as active pharmaceutical ingredients (APIs). However, the compositions disclosed herein are suitable for encapsulating the delivery of other lipophilic anesthetic and non-anesthetic APIs known in the art. The compositions disclosed herein are based on a hydrogel formulation composed of a triblock copolymer component that comprises one or more of PLGA-PEG-PLGA triblock copolymers, wherein any two PLGA-PEG-PLGA triblock copolymers are non-identical in total MW. The triblock copolymer component of the formulations disclosed herein, when dissolved in aqueous media, used forms micelles that allow the polymer to exist in a solution phase at ambient or refrigerated temperatures but forms a soft gel at body temperature.

### SUMMARY OF THE INVENTION

The present disclosure provides a composition of an anesthetic drug of amino amide group formulated in a triblock copolymer component, wherein the composition comprises: a) the anesthetic drug at a concentration of 0.2 to 2% w/v; b) the triblock copolymer component comprises one or more PLGA-PEG-PLGA triblock copolymers at a concentration of 5% to 35% w/v: (i) wherein each Poly-Lactic-co-Glycolic acid (PLGA) polymer block has a molecular weight (MW) of 400 to 2550 Da, (ii) wherein the Polyethylene Glycol (PEG) polymer block has a MW of 1000-3000 Da, (iii) wherein the PLGA comprises 40% to 100% of lactic acid (LA) and 60% to 0% of glycolic acid (GA); c) a polar organic solvent at a concentration of 0% to 20% v/v; and d) a solubility enhancer at a concentration of 0% to 20% v/v, and wherein any two PLGA-PEG-PLGA triblock copolymers are non-identical in total MW.

In some embodiments of the composition provided herein, the formulation comprises: a) lidocaine at a concentration of 1% w/v; b) a PLGA-PEG-PLGA triblock copolymer component at a concentration of 20% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 2% v/v; and d) PEG 400 at a concentration of 15% v/v.

In some embodiments of the composition provided herein, the formulation comprises: a) lidocaine at a concentration of 1% w/v; b) a PLGA-PEG-PLGA triblock copolymer component at a concentration of 17% w/v: (i) wherein the PLGA-PEG-PLGA triblock copolymer component comprises one 1700 Da-1500 Da-1700 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 2% v/v; and d) PEG 400 at a concentration of 15% v/v.

In some embodiments of the composition provided herein, the formulation comprises: a) lidocaine at a concentration of 1% w/v; b) a PLGA-PEG-PLGA triblock copolymer component at a concentration of 23.5% w/v: (i) wherein the PLGA-PEG-PLGA triblock copolymer component comprises one 1700 Da-1500 Da-1700 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 1% v/v; and d) PEG 400 at a concentration of 5% v/v.

In some embodiments of the composition provided herein, the formulation comprises: a) lidocaine at a concentration of 1% w/v; b) a PLGA-PEG-PLGA triblock copolymer component at a concentration of 26% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 75% of lactic acid (LA) and 25% of glycolic acid (GA) (LA:GA ratio of 75:25); and (v) wherein the triblock copolymer component comprises the first PLGA-PEG-PLGA triblock copolymer at a concentration of 54% of the triblock copolymer component and the second PLGA-PEG-PLGA triblock copolymer at a concentration of 46% of the triblock copolymer component; c) DMA at a concentration of 1% v/v; and d) PEG 400 at a concentration of 5% v/v.

In some embodiments of the composition provided herein, the formulation comprises: a) lidocaine at a concentration of 1% w/v; b) a PLGA-PEG-PLGA triblock copolymer component at a concentration of 23.5% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1100 Da-1000 Da-1100 Da triblock copolymer and the second PLGA-PEG-PLGA triblock copolymer is a 1700 Da-1500 Da-1700 Da of PLGA-PEG-PLGA; (iii) wherein the first PLGA-PEG-PLGA triblock copolymer comprises 75% of lactic acid (LA) and 25% of glycolic acid (GA) (LA:GA ratio of 75:25); (iv) wherein the second PLGA-PEG-PLGA triblock copolymer comprises 100% of lactic acid (LA) and 0% of glycolic acid (GA) (LA:GA ratio of 100:0); and (v) wherein the triblock copolymer component comprises the first PLGA-PEG-PLGA triblock copolymer at a concentration of 50% of the triblock copolymer component and the second PLGA-PEG-PLGA triblock copolymer at a concentration of 40% of the triblock copolymer component; c) DMA at a concentration of 1% v/v; and d) PEG 400 at a concentration of 5% v/v.

In some embodiments of the composition provided herein, the formulation comprises: a) bupivacaine at a concentration of 1% w/v; b) a PLGA-PEG-PLGA triblock copolymer component at a concentration of 23% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 10% v/v; and d) PEG 400 at a concentration of 15% v/v.

In some embodiments of the composition provided herein, the formulation comprises: a) bupivacaine at a concentration of 0.5% w/v; b) a PLGA-PEG-PLGA triblock copolymer component at a concentration of 17% w/v: (i) wherein the PLGA-PEG-PLGA triblock copolymer component comprises one 1100 Da-1000 Da-1100 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 5% v/v; and d) PEG 400 at a concentration of 15% v/v.

In some embodiments of the composition provided herein, the formulation comprises: a) bupivacaine at a concentration of 0.5% w/v; b) a PLGA-PEG-PLGA triblock copolymer component at a concentration of 24% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 75% of lactic acid (LA) and 25% of glycolic acid (GA) (LA:GA ratio of 75:25); and (v) wherein the triblock copolymer component comprises the first PLGA-PEG-PLGA triblock copolymer at a concentration of 78% of the triblock copolymer component and the second PLGA-PEG-PLGA triblock copolymer at a concentration of 22% of the triblock copolymer component; c) DMA at a concentration of 5% v/v; and d) PEG 400 at a concentration of 10% v/v.

In some embodiments of the composition provided herein, the formulation comprises: a) bupivacaine at a concentration of 0.5% w/v; b) a PLGA-PEG-PLGA triblock copolymer component at a concentration of 20% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1700 Da-1500 Da-1700 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 100% of lactic acid (LA) and 0% of glycolic acid (GA) (LA:GA ratio of 100:0); and (v) wherein the triblock copolymer component comprises the first PLGA-PEG-PLGA triblock copolymer at a concentration of 60% of the triblock copolymer component and the second PLGA-PEG-PLGA triblock copolymer at a concentration of 40% of the triblock copolymer component; c) DMA at a concentration of 5% v/v; and d) PEG 400 at a concentration of 15% v/v.

In some embodiments of the composition provided herein, the formulation comprises: a) ropivacaine at a concentration of 0.4% w/v; b) a PLGA-PEG-PLGA triblock copolymer component at a concentration of 23% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 10% v/v; and d) PEG 400 at a concentration of 15% v/v.

In some embodiments of the composition provided herein, the formulation comprises: a) ropivacaine at a concentration of 0.4% w/v; b) a PLGA-PEG-PLGA triblock copolymer component at a concentration of 17% w/v: (i) wherein the PLGA-PEG-PLGA triblock copolymer component comprises one 1100 Da-1000 Da-1100 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 5% v/v; and d) PEG 400 at a concentration of 15% v/v.

In some embodiments of the composition provided herein, the formulation comprises: a) ropivacaine at a concentration of 0.4% w/v; b) a PLGA-PEG-PLGA triblock copolymer component at a concentration of 24% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 75% of lactic acid (LA) and 25% of glycolic acid (GA) (LA:GA ratio of 75:25); and (v) wherein the triblock copolymer component comprises the first PLGA-PEG-PLGA triblock copolymer at a concentration of 78% of the triblock copolymer component and the second PLGA-PEG-PLGA triblock copolymer at a concentration of 22% of the triblock copolymer component; c) DMA at a concentration of 5% v/v; and d) PEG 400 at a concentration of 10% v/v.

In some embodiments of the composition provided herein, the formulation comprises: a) ropivacaine at a concentration of 0.4% w/v; b) a PLGA-PEG-PLGA triblock copolymer component at a concentration of 20% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1700 Da-1500 Da-1700 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 100% of lactic acid (LA) and 0% of glycolic acid (GA) (LA:GA ratio of 100:0); and (v) wherein the triblock copolymer component comprises the first PLGA-PEG-PLGA triblock copolymer at a concentration of 60% of the triblock copolymer component and the second PLGA-PEG-PLGA triblock copolymer at a concentration of 40% of the triblock copolymer component; c) DMA at a concentration of 5% v/v; and d) PEG 400 at a concentration of 15% v/v.

The present disclosure also provides a method of prevention or treatment for post-surgical pain in a subject in need thereof, wherein the method comprises administering an effective amount of any of the compositions disclosed herein.

The present disclosure also provides a method of prevention or treatment for pain in a subject in need thereof, wherein the method comprises administering an effective amount of any of the compositions disclosed herein.

The present disclosure provides a method of manufacturing any of the compositions disclosed herein, the method comprising: i) combining and dissolving: a) an amount of the PLGA-PEG-PLGA triblock polymer; b) an amount of water; c) an amount of the anesthetic drug; d) an amount of the polar organic solvent; and e) an amount of the solubility enhancer; to form a mixture; ii) stirring the mixture of (i) at 1-30° C.; iii) filtering the clear solution of (ii) through a sterile filter; and iv) collecting and freezing the filtered solution of (iii) at ≤−20° C.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF DRAWINGS

FIG. 1 is a picture depicting the visual changes to P1 polymer only formulations pre and post heating at 40° C. The visual sol-gel transition of samples of lidocaine, bupivacaine, and ropivacaine formulations A-H from Table 2, as indicated, after heating at 40° C. till opacity is reached or for 20 minutes, and then inverting, is depicted.

FIG. 2 is a graph depicting the IVR profiles of initial formulations amended to 100% at max release. Cumulative drug release profile of the four formulations B, D, G, and H from Table 2 as indicated, is depicted as percentage drug release is shown on the y-axis and time course of the release profile is shown on x-axis.

FIG. 3 is a picture depicting visual changes to formulations with different polymers pre and post heating at 40° C. The visual sol-gel transition of samples of lidocaine, bupivacaine, and ropivacaine formulations I-Q from Table 3, as indicated, after heating at 40° C. till opacity is reached or for 20 minutes, and then inverting, is depicted.

FIG. 4 is a graph depicting the IVR profiles of formulations with different polymer compositions amended to 100% at max release. Cumulative drug release profile of the nine formulations I-Q from Table 3 as indicated, is depicted as percentage drug release is shown on the y-axis and time course of the release profile is shown on x-axis.

FIG. 5 is a picture depicting visual changes to lidocaine formulations with decreased PEG 400 pre and post heating at 40° C. The visual sol-gel transition of samples of lidocaine, bupivacaine, and ropivacaine formulations A-H from Table 4, as indicated, after heating at 40° C. till opacity is reached or for 20 minutes, and then inverting, is depicted.

FIG. 6 is a plot depicting IVR profiles of lidocaine formulations with decreased PEG 400. Cumulative drug release profile of the nine formulations R-T from Table 4 as indicated, is depicted as percentage drug release is shown on the y-axis and time course of the release profile is shown on x-axis.

### DETAILED DESCRIPTION OF THE INVENTION

Bupivacaine (chemical name: 1-butyl-N-(2, 6-dimethylphenyl)piperidine-2-carboxamide; molecular formula: C18H28N2O; molecular weight (MW): 288.4 g/mol) is an amide-type, long-acting local anesthetic. Bupivacaine reversibly binds to specific sodium ion channels in the neuronal membrane, resulting in a decrease in the voltage-dependent membrane permeability to sodium ions and membrane stabilization; inhibition of depolarization and nerve impulse conduction; and a reversible loss of sensation. Bupivacaine is also marketed as 2180-92-9, DL-Bupivacaine, Marcaine, 38396-39-3, Bupivacaina, Sensorcaine, Exparel, Anekain and Carbostesin.

Lidocaine (chemical name: 2-(diethylamino)-N-(2, 6-dimethylphenyl)acetamide; molecular formula; C14H22N₂O; MW: 234.34 g/mol) is a synthetic aminoethylamide with local anesthetic and antiarrhythmic properties. Lidocaine stabilizes the neuronal membrane by binding to and inhibiting voltage-gated sodium channels, thereby inhibiting the ionic fluxes required for the initiation and conduction of impulses and effecting local anesthesia. It has a role as a local anesthetic, an anti-arrhythmia drug, an environmental contaminant, a xenobiotic and a drug allergen. Lidocaine is also marketed under the names 137-58-6, Lignocaine, Xylocaine, Lidoderm, Anestacon, Esracaine, Duncaine and Alphacaine.

Ropivacaine (chemical name: (2S)—N-(2, 6-dimethylphenyl)-1-propylpiperidine-2-carboxamide; molecular formula: C17H26N2O; MW: 274.4 g/mol) is a local anesthetic of the amide type with analgesic activity. Ropivacaine binds to voltage-gated sodium ion channels in the neuronal membrane, thereby preventing the permeability of sodium ions and resulting in a stabilization of the neuronal membrane and inhibition of depolarization; nerve impulse generation and propagation are blocked, resulting in a reversible loss of sensation. Ropivacaine is also marketed under the names 84057-95-4, (S)-Ropivacaine, Naropin, Ropivacaine hydrochloride, Ropivacaine, Ropivacaina, Ropivacainum, Ropivacaina and Ropivacainum.

Lidocaine, bupivacaine and ropivacaine are commonly used local anesthetics that are chemically referred to as aminoethylamides, amino amide group or amide local anesthetics. These local anesthetics have been shown to be effective and are used widely in preventing or reducing pain from minor surgery, incisions, biopsies, dental and obstetrical procedures and pain from wounds. They are usually given as a single injection locally into a lesion or the area of incision but can be given as infusions for hours or for several days by epidural or wound-based catheters. Local anesthetic infusions can also be used for postoperative pain management. Local anesthetics have variable durations of action, and short acting versions are often given with epinephrine, which decreases the rate of absorption limiting systemic exposure and prolonging the duration of action. Side effects of these local anesthetics are usually dose related and occur mostly as a result of systemic administration or exposure. They include neurological symptoms such as drowsiness, tinnitus, dizziness and twitching as well as gastrointestinal effects such as nausea, vomiting and constipation. Cardiovascular depression can also occur and ventricular arrhythmias, especially with higher doses. Thus, there is a need in the art for compositions and methods of safe and efficacious delivery or administration of lidocaine, bupivacaine and ropivacaine for pain management. The present disclosure addresses these needs.

A thermosensitive hydrogel technology is disclosed herein for the delivery of a lidocaine, bupivacaine and ropivacaine for postsurgical pain management. The hydrogel is primarily composed of a PLGA-PEG-PLGA triblock copolymer component comprising one or more PLGA-PEG-PLGA triblock copolymers, wherein any two PLGA-PEG-PLGA triblock copolymers are non-identical in total molecular weight (MW). The triblock polymer upon dissolution in water self-assembles into nano-sized micelles. At ambient temperature, the formulation exists in a solution phase comprised of polymeric micelles which can be loaded with drug. Upon warming to body temperature, interactions between the hydrophobic domains of the micelles become thermodynamically favored, and the micelles form a network resulting in a phase transition from micellar solution into a viscous gel with a consistency similar to that of petroleum jelly. This process is fully reversible upon cooling. This solution to gel phase transformation allows for ease of injection as a solution, with quick conversion to a viscous gel phase once administered allowing for physical depot residency and controlled drug release by simple drug diffusion. A hydrophobic drug can be encapsulated within the hydrophobic core of micelles while the formulation is still liquid (below critical gelation temperature). Encapsulated drug then releases over several days from the gel depot of the formulation.

The structure of lidocaine is well known in the art, for example in Hermanns H. et al., British Journal of Anaesthesia, Volume 123, Issue 3, September 2019, Pages 335-349, and Weinberg L. et al., World J Anesthesiol 2015 Jul. 27; 4(2): 17-29, which are incorporated herein by reference. The structure of bupivacaine is well known in the art, for example in Babst C. R. et al., Anesth Prog. 1978 May-June; 25(3): 87-91, which is incorporated herein by reference. The structure of ropivacaine is well known in the art, for example in McClure J. H. et al., British Journal of Anaesthesia 1996; 76: 300-307, which is incorporated herein by reference.

The present disclosure provides a composition of an anesthetic drug of amino amide group formulated in a PLGA-PEG-PLGA triblock copolymer component, wherein the composition comprises: a) a concentration of the anesthetic drug; b) the triblock copolymer component, wherein the triblock copolymer component comprises one or more PLGA-PEG-PLGA triblock copolymers; c) a polar organic solvent; and d) a solubility enhancer, wherein any two PLGA-PEG-PLGA triblock copolymers are non-identical in total MW. In the compositions of the present disclosure, the balance of compositional mass comprises water.

The present disclosure provides a composition of an anesthetic drug of amino amide group formulated in a triblock copolymer component, wherein the composition comprises: a) the anesthetic drug at a concentration of 0.2 to 2% w/v; b) the triblock copolymer component comprises one or more PLGA-PEG-PLGA triblock copolymers at a concentration of 5% to 35% w/v: (i) wherein each Poly-Lactic-co-Glycolic acid (PLGA) polymer block has a molecular weight (MW) of 400 to 2550 Da, (ii) wherein the Polyethylene Glycol (PEG) polymer block has a MW of 1000-3000 Da, (iii) wherein the PLGA comprises 40% to 100% of lactic acid (LA) and 60% to 0% of glycolic acid (GA); c) a polar organic solvent at a concentration of 0% to 20% v/v; and d) a solubility enhancer at a concentration of 0% to 20% v/v, and wherein any two PLGA-PEG-PLGA triblock copolymers are non-identical in total MW.

The composition can comprise one or more PLGA-PEG-PLGA triblock copolymers with each PLGA polymer block of a MW of 400 to 2550 Da. The composition can comprise one or more PLGA-PEG-PLGA triblock copolymers with each PLGA polymer block of a MW of 400 to 750 Da. The composition can one or more PLGA-PEG-PLGA triblock copolymers with each PLGA polymer block of a MW of 750 to 1000 Da. The composition can comprise one or more PLGA-PEG-PLGA triblock copolymers with each PLGA polymer block of a MW of 1000 to 1250 Da. The composition can comprise one or more PLGA-PEG-PLGA triblock copolymers with each PLGA polymer block of a MW of 1250 to 1500 Da. The composition can comprise one or more PLGA-PEG-PLGA triblock copolymers with each PLGA polymer block of a MW of 1500 to 1750 Da. The composition can comprise one or more PLGA-PEG-PLGA triblock copolymers with each PLGA polymer block of a MW of 1750 to 2000 Da. The composition can comprise one or more PLGA-PEG-PLGA triblock copolymers with each PLGA polymer block of a MW of 2000 to 2250 Da. The composition can comprise one or more PLGA-PEG-PLGA triblock copolymers with each PLGA polymer block of a MW of 2250 to 2500 Da. The composition can comprise one or more PLGA-PEG-PLGA triblock copolymers with each PLGA polymer block of a MW of 2500 to 2550 Da. The composition can comprise one or more PLGA-PEG-PLGA triblock copolymers with each PLGA polymer block of a MW of 400 to 1000 Da. The composition can comprise one or more PLGA-PEG-PLGA triblock copolymers with each PLGA polymer block blocks of a MW of 1000 to 2450 Da. The composition can comprise one or more PLGA-PEG-PLGA triblock copolymers with each PLGA polymer block of a MW of 1400 to 2450 Da. The composition can comprise one or more PLGA-PEG-PLGA triblock copolymers with each PLGA polymer block of a MW of 1500 to 2250 Da. The composition can comprise one or more PLGA-PEG-PLGA triblock copolymers with each PLGA polymer block of a MW of 1000 Da to 1500 Da. The composition can comprise one or more PLGA-PEG-PLGA triblock copolymers with each PLGA polymer block of a MW of 1100 Da to 2000 Da. The composition can comprise one or more PLGA-PEG-PLGA triblock copolymers with each PLGA polymer block of a MW of 1500-2000 Da. The composition can comprise one or more PLGA-PEG-PLGA triblock copolymers with each PLGA polymer block of a MW of 1100 Da to 1500 Da. The composition can comprise one or more PLGA-PEG-PLGA triblock copolymers with each PLGA polymer block of a MW of 1200 Da to 1800 Da. The composition can comprise one or more PLGA-PEG-PLGA triblock copolymers with each PLGA polymer block of a MW of 1000 Da to 1800 Da.

The composition can comprise one or more PLGA-PEG-PLGA triblock copolymers with each PLGA polymer block of a MW of 400 Da. The composition can one or more PLGA-PEG-PLGA triblock copolymers with each PLGA polymer block of a MW of 750 Da. The composition can comprise one or more PLGA-PEG-PLGA triblock copolymers with each PLGA polymer block of a MW of 1000 Da. The composition can comprise one or more PLGA-PEG-PLGA triblock copolymers with each PLGA polymer block of a MW of 1100 Da. The composition can comprise one or more PLGA-PEG-PLGA triblock copolymers with each PLGA polymer block of a MW of 1400 Da. The composition can comprise one or more PLGA-PEG-PLGA triblock copolymers with each PLGA polymer block of a MW of 1500 Da. The composition can comprise one or more PLGA-PEG-PLGA triblock copolymers with each PLGA polymer block of a MW of 1700 Da. The composition can comprise one or more PLGA-PEG-PLGA triblock copolymers with each PLGA polymer block of a MW of 2000 Da. The composition can comprise one or more PLGA-PEG-PLGA triblock copolymers with each PLGA polymer block of a MW of 2450 Da. The composition can comprise one or more PLGA-PEG-PLGA triblock copolymers with each PLGA polymer block of a MW of 2550 Da.

The composition can comprise one or more PLGA-PEG-PLGA triblock copolymers with the PEG polymer block of a MW of 1000-3000 Da. The composition can comprise one or more PLGA-PEG-PLGA triblock copolymers with the PEG polymer block of a MW of 1000-1500 Da. The composition can comprise one or more PLGA-PEG-PLGA triblock copolymers with the PEG polymer block of a MW of 1500-2000 Da. The composition can comprise one or more PLGA-PEG-PLGA triblock copolymers with the PEG polymer block of a MW of 2000-2500 Da. The composition can comprise one or more PLGA-PEG-PLGA triblock copolymers with the PEG polymer block of a MW of 2500-3000 Da. The composition can comprise one or more PLGA-PEG-PLGA triblock copolymers with the PEG polymer block of a MW of 1000-1300 Da. The composition can comprise one or more PLGA-PEG-PLGA triblock copolymers with the PEG polymer block of a MW of 1300-1600 Da. The composition can comprise one or more PLGA-PEG-PLGA triblock copolymers with the PEG polymer block of a MW of 1600-1900 Da. The composition can comprise a one or more PLGA-PEG-PLGA triblock copolymers with the PEG polymer block of a MW of 1900-2200 Da. The composition can comprise one or more PLGA-PEG-PLGA triblock copolymers with the PEG polymer block of a MW of 2200-2500 Da. The composition can comprise one or more PLGA-PEG-PLGA triblock copolymers with the PEG polymer block of a MW of 2500-2750 Da. The composition can comprise one or more PLGA-PEG-PLGA triblock copolymers with the PEG polymer block of a MW of 2750-3000 Da. The composition can comprise one or more PLGA-PEG-PLGA triblock copolymers with the PEG polymer block of a MW of 1000-1600 Da. The composition can comprise one or more PLGA-PEG-PLGA triblock copolymers with the PEG polymer block of a MW of 1400-1600 Da. The composition can comprise one or more PLGA-PEG-PLGA triblock copolymers with the PEG polymer block of a MW of 1600-2000 Da. The composition can comprise one or more PLGA-PEG-PLGA triblock copolymers with the PEG polymer block of a MW of 2000-2400 Da. The composition can comprise one or more PLGA-PEG-PLGA triblock copolymers with the PEG polymer block of a MW of 2000-2800 Da. The composition can comprise one or more PLGA-PEG-PLGA triblock copolymers with the PEG polymer block of a MW of 2800-3000 Da. The composition can comprise one or more PLGA-PEG-PLGA triblock copolymers with the PEG polymer block of a MW of 1000-2000 Da. The composition can comprise a PLGA-PEG-PLGA triblock copolymer with the PEG polymer block of a MW of 1200-1500 Da. The composition can comprise a PLGA-PEG-PLGA triblock copolymer with the PEG polymer block of a MW of 1500-1800 Da. The composition can comprise a PLGA-PEG-PLGA triblock copolymer with the PEG polymer block of a MW of 1800-2000 Da.

The composition can comprise one or more PLGA-PEG-PLGA triblock copolymers with the PEG polymer block of a MW of 1000 Da. The composition can comprise one or more PLGA-PEG-PLGA triblock copolymers with the PEG polymer block of a MW of 1250 Da. The composition can comprise one or more PLGA-PEG-PLGA triblock copolymers with the PEG polymer block of a MW of 1500 Da. The composition can comprise one or more PLGA-PEG-PLGA triblock copolymers with the PEG polymer block of a MW of 1850 Da. The composition can comprise one or more PLGA-PEG-PLGA triblock copolymers with the PEG polymer block of a MW of 2000 Da. The composition can comprise a PLGA-PEG-PLGA triblock copolymer with the PEG polymer block of a MW of 3000 Da.

The composition can comprise a PLGA polymer that has 40% to 100% of lactic acid (LA) and 60% to 0% of glycolic acid (GA). The composition can comprise a PLGA polymer that has 50% to 90% of lactic acid and 50% to 10% glycolic acid. The composition can comprise a PLGA polymer that has 50% to 80% of lactic acid and 50% to 20% glycolic acid. The composition can comprise a PLGA polymer that has 50% to 75% of lactic acid and 50% to 25% of glycolic acid. The composition can comprise a PLGA polymer that has 50% to 70% of lactic acid and 50% to 30% of glycolic acid. The composition can comprise a PLGA polymer that has 50% to 65% lactic acid and 50% to 35% of glycolic acid. The composition can comprise a PLGA polymer that has 50% to 60% lactic acid and 50% to 40% of glycolic acid.

The composition can comprise one or more PLGA-PEG-PLGA triblock copolymers with the PLGA polymer that has 50% of lactic acid and 50% of glycolic acid (LA:GA ratio of 50:50). The composition can comprise a PLGA polymer that has 40% of lactic acid and 60% of glycolic acid (LA:GA ratio of 40:60). The composition can comprise a PLGA polymer that has 43% of lactic acid and 57% of glycolic acid (LA:GA ratio of 43:57). The composition can comprise a PLGA polymer that has 75% of lactic acid and 25% of glycolic acid (LA:GA ratio of 75:25). The composition can comprise a PLGA polymer that has 90% of lactic acid and 10% of glycolic acid (LA:GA ratio of 90:10). The composition can comprise a PLGA polymer that has 100% of lactic acid and 0% of glycolic acid (LA:GA ratio of 100:0).

The composition can comprise a triblock copolymer component that comprises a first PLGA-PEG-PLGA triblock copolymer and a second PLA-PEG-PLA triblock copolymer, wherein the PLGA of the first PLGA-PEG-PLGA triblock copolymer comprises 50% of lactic acid (LA) and 50% of glycolic acid (GA) (LA:GA ratio of 50:50), and the PLGA of the second PLGA-PEG-PLGA triblock copolymer comprises 75% of lactic acid (LA) and 25% of glycolic acid (GA) (LA:GA ratio of 75:25).

The composition can comprise a triblock copolymer component that comprises a first PLGA-PEG-PLGA triblock copolymer and a second PLA-PEG-PLA triblock copolymer, wherein the first PLGA-PEG-PLGA triblock copolymer has a PLGA polymer block of MW of 1200-1500 Da and a PEG polymer block of MW of 1500-2000 Da, and the second PLGA-PEG-PLGA triblock copolymer has a PLGA polymer block of MW of 1000-1200 Da and a PEG polymer block of MW of 1000-1500 Da.

The composition can comprise a triblock copolymer component that comprises a first PLGA-PEG-PLGA triblock copolymer and a second PLA-PEG-PLA triblock copolymer, wherein the first PLGA-PEG-PLGA triblock copolymer is 1400 Da-1500 Da-1400 Da of PLGA-PEG-PLGA, and the second PLGA-PEG-PLGA triblock copolymer is 1100 Da-1000 Da-1100 Da of PLGA-PEG-PLGA.

The composition can comprise a triblock copolymer component that comprises a first PLGA-PEG-PLGA triblock copolymer and a second PLA-PEG-PLA triblock copolymer, wherein the triblock copolymer component comprises the first PLGA-PEG-PLGA triblock copolymer at a concentration of 50%-80% of the triblock copolymer component, and the second PLGA-PEG-PLGA triblock copolymer at a concentration of 20-50% of the triblock copolymer component.

The composition can comprise a triblock copolymer component that comprises a first PLGA-PEG-PLGA triblock copolymer and a second PLA-PEG-PLA triblock copolymer, wherein the PLGA of the first PLGA-PEG-PLGA triblock copolymer comprises 50% of lactic acid (LA) and 50% of glycolic acid (GA) (LA:GA ratio of 50:50), and the PLGA of the second PLGA-PEG-PLGA triblock copolymer comprises 100% of lactic acid (LA) and 0% of glycolic acid (GA) (LA:GA ratio of 100:0).

The composition can comprise a triblock copolymer component that comprises a first PLGA-PEG-PLGA triblock copolymer and a second PLA-PEG-PLA triblock copolymer, wherein the first PLGA-PEG-PLGA triblock copolymer has a PLGA polymer block of MW of 1200-1500 Da and a PEG polymer block of MW of 1500-2000 Da, and the second PLGA-PEG-PLGA triblock copolymer has a PLGA polymer block of MW of 1500-2000 Da and a PEG polymer block of MW of 1000-1500 Da.

The composition can comprise a triblock copolymer component that comprises a first PLGA-PEG-PLGA triblock copolymer and a second PLA-PEG-PLA triblock copolymer, wherein the first PLGA-PEG-PLGA triblock copolymer is 1400 Da-1500 Da-1400 Da of PLGA-PEG-PLGA, and the second PLGA-PEG-PLGA triblock copolymer is 1700 Da-1500 Da-1700 Da.

The composition can comprise a triblock copolymer component that comprises a first PLGA-PEG-PLGA triblock copolymer and a second PLA-PEG-PLA triblock copolymer, wherein the triblock copolymer component comprises the first PLGA-PEG-PLGA triblock copolymer at a concentration of 50% to 70% of the triblock copolymer component and the second PLGA-PEG-PLGA triblock copolymer at a concentration of 30% to 50% of the triblock copolymer component.

The composition can comprise a triblock copolymer component that comprises a first PLGA-PEG-PLGA triblock copolymer and a second PLA-PEG-PLA triblock copolymer, wherein the triblock copolymer component comprise a first PLGA-PEG-PLGA triblock copolymer and a second PLGA-PEG-PLGA triblock copolymer, wherein the PLGA of the first PLGA-PEG-PLGA triblock copolymer comprises 75% of lactic acid (LA) and 25% of glycolic acid (GA) (LA:GA ratio of 75:25), and the PLGA of the second PLGA-PEG-PLGA triblock copolymer comprises 100% of lactic acid (LA) and 0% of glycolic acid (GA) (LA:GA ratio of 100:0).

The composition can comprise a triblock copolymer component that comprises a first PLGA-PEG-PLGA triblock copolymer and a second PLA-PEG-PLA triblock copolymer, wherein the first PLGA-PEG-PLGA triblock copolymer has a PLGA polymer block of MW of 1000-1200 Da and a PEG polymer block of MW of 1000-1500 Da, and the second PLGA-PEG-PLGA triblock copolymer has a PLGA polymer block of MW of 1500-2000 Da and a PEG polymer block of MW of 1000-1500 Da.

The composition can comprise a triblock copolymer component that comprises a first PLGA-PEG-PLGA triblock copolymer and a second PLA-PEG-PLA triblock copolymer, wherein the first PLGA-PEG-PLGA triblock copolymer is 1100 Da-1000 Da-1100 Da of PLGA-PEG-PLGA, and the second PLGA-PEG-PLGA triblock copolymer is 1700 Da-1500 Da-1700 Da of PLGA-PEG-PLGA.

The composition can comprise a triblock copolymer component that comprises a first PLGA-PEG-PLGA triblock copolymer and a second PLA-PEG-PLA triblock copolymer, wherein the triblock copolymer component comprises the first PLGA-PEG-PLGA triblock copolymer at a concentration of 50% to 80% of the triblock copolymer component and the second PLGA-PEG-PLGA triblock copolymer at a concentration of 20% to 50% of the triblock copolymer component.

The composition can comprise a triblock copolymer component that comprises one PLGA-PEG-PLGA triblock copolymer, wherein the PLGA-PEG-PLGA triblock copolymer comprises 50% of lactic acid (LA) and 50% of glycolic acid (GA) (LA:GA ratio of 50:50).

The composition can comprise a triblock copolymer component that comprises one PLGA-PEG-PLGA triblock copolymer, wherein the PLGA-PEG-PLGA triblock copolymer has a PLGA polymer block of MW of 1200-1500 Da and a PEG polymer block of MW of 1500-2000 Da.

The composition can comprise a triblock copolymer component that comprises one PLGA-PEG-PLGA triblock copolymer, wherein the PLGA-PEG-PLGA triblock copolymer is 1400 Da-1500 Da-1400 Da of PLGA-PEG-PLGA.

The composition can comprise a triblock copolymer component that comprises one PLGA-PEG-PLGA triblock copolymer, wherein the triblock copolymer component comprise one PLGA-PEG-PLGA triblock copolymer, wherein the PLGA-PEG-PLGA triblock copolymer comprises 75% of lactic acid (LA) and 25% of glycolic acid (GA) (LA:GA ratio of 75:25).

The composition can comprise a triblock copolymer component that comprises one PLGA-PEG-PLGA triblock copolymer, wherein the PLGA-PEG-PLGA triblock copolymer has a PLGA polymer block of MW of 1000-1200 Da and a PEG polymer block of MW of 1000-1500 Da.

The composition can comprise a triblock copolymer component that comprises one PLGA-PEG-PLGA triblock copolymer, wherein the PLGA-PEG-PLGA triblock copolymer is 1100 Da-1000 Da-1100 Da of PLGA-PEG-PLGA.

The composition can comprise a triblock copolymer component that comprises one PLGA-PEG-PLGA triblock copolymer, wherein the triblock copolymer component comprises a PLGA-PEG-PLGA triblock copolymer, wherein the PLGA-PEG-PLGA triblock copolymer comprises 100% of lactic acid (LA) and 0% of glycolic acid (GA) (LA:GA ratio of 100:0).

The composition can comprise a triblock copolymer component that comprises one PLGA-PEG-PLGA triblock copolymer, wherein the PLGA-PEG-PLGA triblock copolymer has a PLA polymer block of MW of 1500-2000 Da and a PEG polymer block of MW of 1000-1500 Da.

The composition can comprise a triblock copolymer component that comprises one PLGA-PEG-PLGA triblock copolymer, wherein the PLGA-PEG-PLGA triblock copolymer is 1700 Da-1500 Da-1700 Da of PLGA-PEG-PLGA.

The composition can comprise the anesthetic drug of amino amide group at a concentration of about 0.2 to about 2% w/w. The composition can comprise the anesthetic drug of amino amide group at a concentration of 0.2 to 2% w/w. The composition can comprise the anesthetic drug of amino amide group at a concentration of 0.2 to 1.8% w/v. The composition can comprise the anesthetic drug of amino amide group at a concentration of 0.2 to 1.5% w/v. The composition can comprise the anesthetic drug of amino amide group at a concentration of 0.65 to 1.3% w/v. The composition can comprise the anesthetic drug of amino amide group at a concentration of 0.2 to 1% w/w. The composition can comprise the anesthetic drug of amino amide group at a concentration of 0.3 to 1% w/v. The composition can comprise the anesthetic drug of amino amide group at a concentration of 0.4 to 1.0% w/v. The composition can comprise the anesthetic drug of amino amide group at a concentration of 0.5 to 1% w/v. The composition can comprise the anesthetic drug of amino amide group at a concentration of 0.3 to 0.5% w/v. The composition can comprise the anesthetic drug of amino amide group at a concentration of 0.3 to 0.4% w/v. The composition can comprise the anesthetic drug of amino amide group at a concentration of 0.2% w/v. The composition can comprise the anesthetic drug of amino amide group at a concentration of 0.3% w/v. The composition can comprise the anesthetic drug of amino amide group at a concentration of 0.4% w/v. The composition can comprise the anesthetic drug of amino amide group at a concentration of 0.5% w/v. The composition can comprise the anesthetic drug of amino amide group at a concentration of 0.6% w/v. The composition can comprise the anesthetic drug of amino amide group at a concentration of 0.65% w/v. The composition can comprise the anesthetic drug of amino amide group at a concentration of 0.8% w/v. The composition can comprise the anesthetic drug of amino amide group at a concentration of 1% w/v. The composition can comprise the anesthetic drug of amino amide group at a concentration of 1.3% w/v. The composition can comprise the anesthetic drug of amino amide group at a concentration of 2% w/v.

The composition can comprise the anesthetic drug of amino amide group that can be any one of a bupivacaine, lidocaine, and ropivacaine. The composition can comprise lidocaine. The composition can comprise bupivacaine. The composition can comprise ropivacaine.

The composition can comprise lidocaine at a concentration of about 0.2 to about 2% w/v. The composition can comprise lidocaine at a concentration of 0.2 to 2% w/v. The composition can lidocaine at a concentration of 0.2 to 1.8% w/v. The composition can comprise lidocaine at a concentration of 0.2 to 1.5% w/v. The composition can comprise lidocaine at a concentration of 0.65 to 1.3% w/v. The composition can comprise lidocaine at a concentration of 0.2 to 1% w/v. The composition can comprise lidocaine at a concentration of 0.3 to 1% w/v. The composition can comprise lidocaine at a concentration of 0.4 to 1.0% w/v. The composition can comprise lidocaine at a concentration of 0.5 to 1% w/v. The composition can comprise lidocaine at a concentration of 0.3 to 0.5% w/v. The composition can comprise lidocaine at a concentration of 0.3 to 0.4% w/v. The composition can comprise lidocaine at a concentration of 0.2% w/v. The composition can comprise lidocaine at a concentration of 0.3% w/v. The composition can comprise lidocaine at a concentration of 0.4% w/v. The composition can comprise lidocaine at a concentration of 0.5% w/v. The composition can comprise lidocaine at a concentration of 0.6% w/v. The composition can comprise lidocaine at a concentration of 0.65% w/v. The composition can comprise lidocaine at a concentration of 0.8% w/v. The composition can comprise lidocaine at a concentration of 1% w/v. The composition can comprise lidocaine at a concentration of 1.3% w/v. The composition can comprise lidocaine at a concentration of 2% w/v.

The composition can comprise bupivacaine at a concentration of about 0.2 to about 2% w/v. The composition can comprise bupivacaine at a concentration of 0.2 to 2% w/v. The composition can comprise bupivacaine at a concentration of 0.2 to 1.8% w/v. The composition can comprise bupivacaine at a concentration of 0.2 to 1.5% w/v. The composition can comprise bupivacaine at a concentration of 0.65 to 1.3% w/v. The composition can comprise bupivacaine at a concentration of 0.2 to 1% w/v. The composition can comprise bupivacaine at a concentration of 0.3 to 1% w/v. The composition can comprise bupivacaine at a concentration of 0.4 to 1.0% w/v. The composition can comprise bupivacaine at a concentration of 0.5 to 1% w/v. The composition can comprise bupivacaine at a concentration of 0.3 to 0.5% w/v. The composition can comprise bupivacaine at a concentration of 0.3 to 0.4% w/v. The composition can comprise bupivacaine at a concentration of 0.2% w/v. The composition can comprise bupivacaine at a concentration of 0.3% w/v. The composition can comprise bupivacaine at a concentration of 0.4% w/v. The composition can comprise bupivacaine at a concentration of 0.5% w/v. The composition can comprise bupivacaine at a concentration of 0.6% w/v. The composition can comprise bupivacaine at a concentration of 0.65% w/v. The composition can comprise bupivacaine at a concentration of 0.8% w/v. The composition can comprise bupivacaine at a concentration of 1% w/v. The composition can comprise bupivacaine at a concentration of 1.3% w/v. The composition can comprise bupivacaine at a concentration of 2% w/v.

The composition can comprise ropivacaine at a concentration of about 0.2 to about 2% w/v. The composition can comprise ropivacaine at a concentration of 0.2 to 2% w/v. The composition can comprise ropivacaine at a concentration of 0.2 to 1.8% w/v. The composition can comprise ropivacaine at a concentration of 0.2 to 1.5% w/v. The composition can comprise ropivacaine at a concentration of 0.65 to 1.3% w/v. The composition can comprise ropivacaine at a concentration of 0.2 to 1% w/v. The composition can comprise ropivacaine at a concentration of 0.3 to 1% w/v. The composition can comprise ropivacaine at a concentration of 0.4 to 1.0% w/v. The composition can comprise ropivacaine at a concentration of 0.5 to 1% w/v. The composition can comprise ropivacaine at a concentration of 0.3 to 0.5% w/v. The composition can comprise ropivacaine at a concentration of 0.3 to 0.4% w/v. The composition can comprise ropivacaine at a concentration of 0.2% w/v. The composition can comprise ropivacaine at a concentration of 0.3% w/v. The composition can comprise ropivacaine at a concentration of 0.4% w/v. The composition can comprise ropivacaine at a concentration of 0.5% w/v. The composition can comprise ropivacaine at a concentration of 0.6% w/v. The composition can comprise bupivacaine at a concentration of 0.65% w/v. The composition can comprise ropivacaine at a concentration of 0.8% w/v. The composition can comprise ropivacaine at a concentration of 1% w/v. The composition can comprise ropivacaine at a concentration of 1.3% w/v. The composition can comprise ropivacaine at a concentration of 2% w/v.

The composition can comprise a polar organic solvent that is 0-1% v/v. The composition can comprise a polar organic solvent that is 0-0.5% v/v. The composition can comprise a polar organic solvent that is 0-0.2% v/v. The composition can comprise a polar organic solvent that is 0.1-0.5% v/v. The composition can comprise a polar organic solvent that is 0.5-1% v/v. The composition can comprise a polar organic solvent that is 1-5% v/v. The composition can comprise a polar organic solvent that is 1-3% v/v. The composition can comprise a polar organic solvent that is 1.5-3% v/v. The composition can comprise a polar organic solvent that is 1-2% v/v. The composition can comprise a polar organic solvent that is 1.5-2% v/v.

The composition can comprise a polar organic solvent that is 0.1-20% v/v. The composition can comprise a polar organic solvent that is 0.2-20% v/v. The composition can comprise a polar organic solvent that is 0.5-20% v/v. The composition can comprise a polar organic solvent that is 0.75-20% v/v. The composition can comprise a polar organic solvent that is 1-20% v/v.

The composition can comprise a polar organic solvent that is 1-10% v/v. The composition can comprise a polar organic solvent that is 2-10% v/v. The composition can comprise a polar organic solvent that is 1-10.5% v/v. The composition can comprise a polar organic solvent that is 5-10% v/v. The composition can comprise a polar organic solvent that is 10-20% v/v. The composition can comprise a polar organic solvent that is 10-15% v/v. The composition can comprise a polar organic solvent that is 15-20% v/v.

The composition can comprise a polar organic solvent that is 1% v/v. The composition can comprise a polar organic solvent that is 1.5% v/v. The composition can comprise a polar organic solvent that is 2% v/v. The composition can comprise a polar organic solvent that is 2.5% v/v. The composition can comprise a polar organic solvent that is 3% v/v. The composition can comprise a polar organic solvent that is 3.5% v/v. The composition can comprise a polar organic solvent that is 4% v/v. The composition can comprise a polar organic solvent that is 4.5% v/v. The composition can comprise a polar organic solvent that is 5% v/v. The composition can be without any polar organic solvent.

The composition can comprise a polar organic solvent that is any one of Dimethyl acetamide (DMA), Dimethyl sulfoxide (DMSO), Glycofurol, and N-Methyl-2-Pyrrolidone (NMP). The composition can comprise a polar organic solvent that is DMA.

The composition can comprise a solubility enhancer that is 0-0.5% v/v. The composition can comprise a solubility enhancer that is 0-0.1% v/v. The composition can comprise a solubility enhancer that is 0.1-0.2% v/v. The composition can comprise a solubility enhancer that is 0.2-0.5% v/v. The composition can comprise a solubility enhancer that is 0.5-1% v/v. The composition can comprise a solubility enhancer that is 1-7.5% v/v. The composition can comprise a solubility enhancer that is 1-7.5% v/v. The composition can comprise a solubility enhancer that is 1-10.5% v/v. The composition can comprise a solubility enhancer that is 1-12.5% v/v. The composition can comprise a solubility enhancer that is 1-15% w/w. The composition can comprise a solubility enhancer that is 1-17.5% v/v. The composition can comprise a solubility enhancer that is 1-20% v/v. The composition can comprise a solubility enhancer that is 1.5-20% v/v. The composition can comprise a solubility enhancer that is 2-20% v/v. The composition can comprise a solubility enhancer that is 2.5-20% v/v. The composition can comprise a solubility enhancer that is 3-20% w/w. The composition can comprise a solubility enhancer that is 5-20% v/v. The composition can comprise a solubility enhancer that is 1-5% v/v. The composition can comprise a solubility enhancer that is 1-3% v/v. The composition can comprise a solubility enhancer that is 1-2% v/v. The composition can comprise a solubility enhancer that is 1-1.5% v/v. The composition can comprise a solubility enhancer that is 1.5-2% v/v. The composition can comprise a solubility enhancer that is 1.7-2% v/v. The composition can comprise a solubility enhancer that is 2-2.5% v/v. The composition can comprise a solubility enhancer that is 2-3% v/v. The composition can comprise a solubility enhancer that is 2-5% v/v. The composition can comprise a solubility enhancer that is 3-5% v/v. The composition can comprise a solubility enhancer that is 5-10% v/v. The composition can comprise a solubility enhancer that is 10-15% v/v. The composition can comprise a solubility enhancer that is 15-20% v/v. The composition can comprise a solubility enhancer that is 15-17% v/v. The composition can comprise a solubility enhancer that is 17-20% v/v. The composition can comprise a solubility enhancer that is 5-11% v/v. The composition can comprise a solubility enhancer that is 6-11% v/v. The composition can comprise a solubility enhancer that is 5-7% v/v. The composition can comprise a solubility enhancer that is 7-11% v/v. The composition can comprise a solubility enhancer that is 7-9% v/v. The composition can comprise a solubility enhancer that is 8-11% v/v. The composition can comprise a solubility enhancer that is 9-11% v/v. The composition can comprise a solubility enhancer that is 10-11% v/v. The composition can comprise a solubility enhancer that is 11-15% v/v. The composition can comprise a solubility enhancer that is 11-13% v/v. The composition can comprise a solubility enhancer that is 11-12% v/v. The composition can comprise a solubility enhancer that is 13-16% v/v. The composition can comprise a solubility enhancer that is 16-19% v/v. The composition can comprise a solubility enhancer that is 19-20% v/v.

The composition can comprise a solubility enhancer that is 1.7% v/v. The composition can comprise a solubility enhancer that is 5% v/v. The composition can comprise a solubility enhancer that is 6% v/v. The composition can comprise a solubility enhancer that is 6.2% v/v. The composition can comprise a solubility enhancer that is 6.4% v/v. The composition can comprise a solubility enhancer that is 6.5% v/v. The composition can comprise a solubility enhancer that is 8.3% v/v. The composition can comprise a solubility enhancer that is 8.4% v/v. The composition can comprise a solubility enhancer that is 8.6% v/v. The composition can comprise a solubility enhancer that is 8.7% v/v. The composition can comprise a solubility enhancer that is 8.8% v/v. The composition can comprise a solubility enhancer that is 8.9% v/v. The composition can comprise a solubility enhancer that is 9.1% v/v. The composition can comprise a solubility enhancer that is 9.2% v/v. The composition can comprise a solubility enhancer that is 9.4% v/v. The composition can comprise a solubility enhancer that is 9.6% v/v. The composition can comprise a solubility enhancer that is 10% v/v. The composition can comprise a solubility enhancer that is 10.3% v/v. The composition can comprise a solubility enhancer that is 10.4% v/v. The composition can comprise a solubility enhancer that is 10.5% v/v. The composition can comprise a solubility enhancer that is 10.6% v/v. The composition can comprise a solubility enhancer that is 11% v/v. The composition can comprise a solubility enhancer that is 12% v/v. The composition can comprise a solubility enhancer that is 15% v/v. The composition can comprise a solubility enhancer that is 20% v/v.

The composition can comprise a solubility enhancer that is any one of Propylene glycol (PG), Cremophor EL, Cremophor RH 60, Ethanol, Glycerin, PEG 300, PEG 400, Polysorbates, Vitamin E-TPGS, PLGA-PEG diblock copolymers, hydroxypropyl-β-cyclodextrin, sulfobutyl ether-β-cyclodextrin, poloxamer 188, poloxomer 407, polyvinyl pyrrolidone (PVP), glycerol formal, and Solutol HS. The composition can comprise a solubility enhancer that is polysorbate 80. The composition can comprise a solubility enhancer that is PEG 400.

The composition can comprise a PLGA-PEG-PLGA triblock copolymer component at a concentration of 5-30% w/v. The composition can comprise a PLGA-PEG-PLGA triblock copolymer component at a concentration of 5-25% w/v. The composition can comprise a PLGA-PEG-PLGA triblock copolymer component at a concentration of 5-20% w/v. The composition can comprise a PLGA-PEG-PLGA triblock copolymer component at a concentration of 5-15% w/v. The composition can comprise a PLGA-PEG-PLGA triblock copolymer component at a concentration of 5-10% w/v. The composition can comprise a PLGA-PEG-PLGA triblock copolymer component at a concentration of 10-20% w/v. The composition can comprise a PLGA-PEG-PLGA triblock copolymer component at a concentration of 8-35% w/v. The composition can comprise a PLGA-PEG-PLGA triblock copolymer component at a concentration of 8-30% w/v. The composition can comprise a PLGA-PEG-PLGA triblock copolymer component at a concentration of 8-25% w/v. The composition can comprise a PLGA-PEG-PLGA triblock copolymer component at a concentration of 8%-20% w/v. The composition can comprise a PLGA-PEG-PLGA triblock copolymer component at a concentration of 8-15% w/v. The composition can comprise a PLGA-PEG-PLGA triblock copolymer component at a concentration of 8-10% w/v. The composition can comprise a PLGA-PEG-PLGA triblock copolymer component at a concentration of 5-8% w/v. The composition can comprise a PLGA-PEG-PLGA triblock copolymer component at a concentration of 8%-10% w/v. The composition can comprise a PLGA-PEG-PLGA triblock copolymer component at a concentration of 10-15% w/v. The composition can comprise a PLGA-PEG-PLGA triblock copolymer component at a concentration of 15-20% w/v. The composition can comprise a PLGA-PEG-PLGA triblock copolymer component at a concentration of 20-25% w/v.

The composition can comprise a PLGA-PEG-PLGA triblock copolymer component at a concentration of 10-30% w/v. The composition can comprise a PLGA-PEG-PLGA triblock copolymer component at a concentration of 10-26% w/v. The composition can comprise a PLGA-PEG-PLGA triblock copolymer component at a concentration of 16-26% w/v.

The composition can comprise a PLGA-PEG-PLGA triblock copolymer component at a concentration of 19-20% w/v. The composition can comprise a PLGA-PEG-PLGA triblock copolymer component at a concentration of 19.1-20% w/v. The composition can comprise a PLGA-PEG-PLGA triblock copolymer component at a concentration of 19.2-20% w/v. The composition can comprise a PLGA-PEG-PLGA triblock copolymer component at a concentration of 19.5-20% w/v. The composition can comprise a PLGA-PEG-PLGA triblock copolymer component at a concentration of 19.8-20% w/v. The composition can comprise a PLGA-PEG-PLGA triblock copolymer component at a concentration of 19.0-19.5% w/v. The composition can comprise a PLGA-PEG-PLGA triblock copolymer component at a concentration of 19.0-19.2% w/v. The composition can comprise a PLGA-PEG-PLGA triblock copolymer component at a concentration of 19.2-19.5% w/v. The composition can comprise a PLGA-PEG-PLGA triblock copolymer component at a concentration of 19.5-20% w/v. The composition can comprise a PLGA-PEG-PLGA triblock copolymer component at a concentration of 25-30% w/v. The composition can comprise a PLGA-PEG-PLGA triblock copolymer component at a concentration of 30-35% w/v. The composition can comprise a PLGA-PEG-PLGA triblock copolymer component at a concentration of 25-35% w/v.

The composition can comprise a PLGA-PEG-PLGA triblock copolymer component at a concentration of 17.0% w/v. The composition can comprise a PLGA-PEG-PLGA triblock copolymer component at a concentration of 17.5% w/v. The composition can comprise a PLGA-PEG-PLGA triblock copolymer component at a concentration of 17.0% w/v. The composition can comprise a PLGA-PEG-PLGA triblock copolymer component at a concentration of 18% w/v. The composition can comprise a PLGA-PEG-PLGA triblock copolymer component at a concentration of 18.5% w/v. The composition can comprise a PLGA-PEG-PLGA triblock copolymer component at a concentration of 19.0% w/v. The composition can comprise a PLGA-PEG-PLGA triblock copolymer component at a concentration of 19.1% w/v. The composition can comprise a PLGA-PEG-PLGA triblock copolymer component at a concentration of 19.2% w/v. The composition can comprise a PLGA-PEG-PLGA triblock copolymer component at a concentration of 19.25% w/v. The composition can comprise a PLGA-PEG-PLGA triblock copolymer component at a concentration of 19.3% w/v. The composition can comprise a PLGA-PEG-PLGA triblock copolymer component at a concentration of 19.4% w/v. The composition can comprise a PLGA-PEG-PLGA triblock copolymer component at a concentration of 19.5% w/v. The composition can comprise a PLGA-PEG-PLGA triblock copolymer component at a concentration of 19.6% w/v. The composition can comprise a PLGA-PEG-PLGA triblock copolymer component at a concentration of 19.7% w/v. The composition can comprise a PLGA-PEG-PLGA triblock copolymer component at a concentration of 19.8% w/v. The composition can comprise a PLGA-PEG-PLGA triblock copolymer component at a concentration of 19.9% w/v. The composition can comprise a PLGA-PEG-PLGA triblock copolymer component at a concentration of 20% w/v. The composition can comprise a PLGA-PEG-PLGA triblock copolymer component at a concentration of 20.5% w/v. The composition can comprise a PLGA-PEG-PLGA triblock copolymer component at a concentration of 21% w/v. The composition can comprise a PLGA-PEG-PLGA triblock copolymer component at a concentration of 21.5% w/v. The composition can comprise a PLGA-PEG-PLGA triblock copolymer component at a concentration of 22% w/v. The composition can comprise a PLGA-PEG-PLGA triblock copolymer component at a concentration of 22.5% w/v. The composition can comprise a PLGA-PEG-PLGA triblock copolymer component at a concentration of 23% w/v. The composition can comprise a PLGA-PEG-PLGA triblock copolymer component at a concentration of 23.5% w/v. The composition can comprise a PLGA-PEG-PLGA triblock copolymer component at a concentration of 24% w/v. The composition can comprise a PLGA-PEG-PLGA triblock copolymer component at a concentration of 24.5% w/v. The composition can comprise a PLGA-PEG-PLGA triblock copolymer component at a concentration of 25% w/v. The composition can comprise a PLGA-PEG-PLGA triblock copolymer component at a concentration of 25.5% w/v. The composition can comprise a PLGA-PEG-PLGA triblock copolymer component at a concentration of 26% w/v. The composition can comprise a PLGA-PEG-PLGA triblock copolymer component at a concentration of 26.5% w/v. The composition can comprise a PLGA-PEG-PLGA triblock copolymer component at a concentration of 27% w/v.

The composition can comprise a total molecular weight of any of the one or more triblock copolymers of the triblock copolymer component that is between 2000-7000 Da. The composition can comprise a total molecular weight of any of the one or more triblock copolymers of the triblock copolymer component that is between 2500-7000 Da. The composition can comprise a total molecular weight of any of the one or more triblock copolymers of the triblock copolymer component that is between 3000-7000 Da. The composition can comprise a total molecular weight of any of the one or more triblock copolymers of the triblock copolymer component that is between 3500-7000 Da. The composition can comprise a total molecular weight of any of the one or more triblock copolymers of the triblock copolymer component that is between 4000-7000 Da. The composition can comprise a total molecular weight of any of the one or more triblock copolymers of the triblock copolymer component that is between 4000-5000 Da. The composition can comprise a total molecular weight of any of the one or more triblock copolymers of the triblock copolymer component that is between 5000-6000 Da. The composition can comprise a total molecular weight of any of the one or more triblock copolymers of the triblock copolymer component that is between 6000-7000 Da. The composition can comprise a total molecular weight of any of the one or more triblock copolymers of the triblock copolymer component that is between 5000-7000 Da The composition can comprise a total molecular weight of any of the one or more triblock copolymers of the triblock copolymer component that is between 2600-6400 Da. The composition can comprise a total molecular weight of any of the one or more triblock copolymers of the triblock copolymer component that is between 4300-6400 Da The composition can comprise a total molecular weight of any of the one or more triblock copolymers of the triblock copolymer component that does not exceed more than 6600-7000 Da. The composition can comprise a total molecular weight of any of the one or more triblock copolymers of the triblock copolymer component that is between 3000-3500 Da. The composition can comprise a total molecular weight of any of the one or more triblock copolymers of the triblock copolymer component that is between 3500-4000 Da. The composition can comprise a total molecular weight of any of the one or more triblock copolymers of the triblock copolymer component that is between 4000-4500 Da. The composition can comprise a total molecular weight of any of the one or more triblock copolymers of the triblock copolymer component that is between 4500-5000 Da. The composition can comprise a total molecular weight of any of the one or more triblock copolymers of the triblock copolymer component that is 6000 Da. The composition can comprise a total molecular weight of any of the one or more triblock copolymers of the triblock copolymer component that is less than 6000 Da. The composition can comprise a total molecular weight of any of the one or more triblock copolymers of the triblock copolymer component that is 3000 Da. The composition can comprise a total molecular weight of any of the one or more triblock copolymers of the triblock copolymer component that is 3100 Da. The composition can comprise a total molecular weight of any of the one or more triblock copolymers of the triblock copolymer component that is 4000 Da. The composition can comprise a total molecular weight of any of the one or more triblock copolymers of the triblock copolymer component that is 4300 Da. The composition can comprise a total molecular weight of any of the one or more triblock copolymers of the triblock copolymer component that is 4500 Da. The composition can comprise a total molecular weight of any of the one or more triblock copolymers of the triblock copolymer component that is 4900 Da. The composition can comprise a total molecular weight of any of the one or more triblock copolymers of the triblock copolymer component that is 5000 Da.

The composition can comprise any one of the one or more PLGA-PEG-PLGA triblock copolymers that is 400 Da-3000 Da-400 Da of PLGA-PEG-PLGA The composition can comprise any one of the one or more PLGA-PEG-PLGA triblock copolymers that is 400 Da-3050 Da-400 Da of PLGA-PEG-PLGA. The composition can comprise any one of the one or more PLGA-PEG-PLGA triblock copolymers that is 750 Da-1850 Da-750 Da of PLGA-PEG-PLGA. The composition can comprise any one of the one or more PLGA-PEG-PLGA triblock copolymers that is 750 Da-2500 Da-750 Da of PLGA-PEG-PLGA.

The composition can comprise any one of the one or more PLGA-PEG-PLGA triblock copolymers that is 1000 Da-1000 Da of PLGA-PEG-PLGA. The composition can comprise any one of the one or more PLGA-PEG-PLGA triblock copolymers that is 1500 Da-1500 Da of PLGA-PEG-PLGA. The composition can comprise any one of the one or more PLGA-PEG-PLGA triblock copolymers that is 2000 Da-2000 Da-2000 Da of PLGA-PEG-PLGA. The composition can comprise a PLGA-PEG-PLGA that is 2550 Da-1550 Da-2550 Da of PLGA-PEG-PLGA.

The composition can comprise any one of the one or more PLGA-PEG-PLGA triblock copolymers that is 1600 Da-1500 Da-1600 Da of PLGA-PEG-PLGA. The composition can comprise any one of the one or more PLGA-PEG-PLGA triblock copolymers that is 1600 Da-1000 Da-1600 Da of PLGA-PEG-PLGA. The composition can comprise any one of the one or more PLGA-PEG-PLGA triblock copolymers that is 1100 Da-1000 Da-1100 of PLGA-PEG-PLGA. The composition can comprise any one of the one or more PLGA-PEG-PLGA triblock copolymers that is 1400 Da-1500 Da-1400 of PLGA-PEG-PLGA. The composition can comprise any one of the one or more PLGA-PEG-PLGA triblock copolymers that is 1700 Da-1500 Da-1700 Da of PLGA-PEG-PLGA. The composition can comprise any one of the one or more PLGA-PEG-PLGA triblock copolymers that is 2450 Da-1500 Da-2450 Da of PLGA-PEG-PLGA.

The present disclosure provides a composition of an anesthetic drug of amino amide group formulated in a triblock copolymer component, wherein the composition comprises: a) the anesthetic drug at a concentration of 0.4-1% w/v; b) a PLGA-PEG-PLGA triblock copolymer component at a concentration of 17% w/v: (I) wherein the PLGA-PEG-PLGA triblock copolymer component comprises one 1700 Da-1500 Da-1700 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 2-5% v/v; and d) PEG 400 at a concentration of 15% v/v.

The present disclosure provides a composition of an anesthetic drug of amino amide group formulated in a triblock copolymer component, wherein the composition comprises: a) the anesthetic drug at a concentration at a concentration of 0.3-1% w/v; b) a PLGA-PEG-PLGA triblock copolymer component at a concentration of 20-24% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 75% of lactic acid (LA) and 25% of glycolic acid (GA) (LA:GA ratio of 75:25); and (v) wherein the triblock copolymer component comprises the first PLGA-PEG-PLGA triblock copolymer at a concentration of 78% of the triblock copolymer component and the second PLGA-PEG-PLGA triblock copolymer at a concentration of 22% of the triblock copolymer component; c) DMA at a concentration of 2-5% v/v; and d) PEG 400 at a concentration of 10% v/v. The composition can comprise any one of lidocaine, bupivacaine and ropivacaine.

The present disclosure provides a composition of an anesthetic drug of amino amide group formulated in a triblock copolymer component, wherein the composition comprises: a) the anesthetic drug at a concentration of 0.4-1% w/v; b) a PLGA-PEG-PLGA triblock copolymer component at a concentration of 20% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1700 Da-1500 Da-1700 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 100% of lactic acid (LA) and 0% of glycolic acid (GA) (LA:GA ratio of 100:0); and (v) wherein the triblock copolymer component comprises the first PLGA-PEG-PLGA triblock copolymer at a concentration of 60% of the triblock copolymer component and the second PLGA-PEG-PLGA triblock copolymer at a concentration of 40% of the triblock copolymer component; c) DMA at a concentration of 2-5% v/v; and d) PEG 400 at a concentration of 15% v/v. The composition can comprise any one of lidocaine, bupivacaine and ropivacaine.

The present disclosure provides a composition of lidocaine formulated in a PLGA-PEG-PLGA triblock copolymer component, wherein the composition comprises: a) lidocaine at a concentration of 1% w/v; b) a PLGA-PEG-PLGA triblock copolymer component at a concentration of 20-25% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 2% v/v; and d) PEG 400 at a concentration of 10-15% v/v.

The present disclosure provides a composition of bupivacaine formulated in a PLGA-PEG-PLGA triblock copolymer component, wherein the composition comprises: a) bupivacaine at a concentration of 3-4% w/v; b) a PLGA-PEG-PLGA triblock copolymer component at a concentration of 23-25% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 5-10% v/v; and d) PEG 400 at a concentration of 10-15% v/v.

The present disclosure provides a composition of ropivacaine formulated in a PLGA-PEG-PLGA triblock copolymer component, wherein the composition comprises: a) ropivacaine at a concentration of 3-4% w/v; b) a PLGA-PEG-PLGA triblock copolymer component at a concentration of 23-25% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 5-10% v/v; and d) PEG 400 at a concentration of 10-15% v/v.

The present disclosure provides a composition of lidocaine formulated in a PLGA-PEG-PLGA triblock copolymer component, wherein the composition comprises: a) lidocaine at a concentration of 1% w/v; b) a PLGA-PEG-PLGA triblock copolymer component at a concentration of 20% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 2% v/v; and d) PEG 400 at a concentration of 15% v/v.

The present disclosure provides a composition of lidocaine formulated in a PLGA-PEG-PLGA triblock copolymer component, wherein the composition comprises: a) lidocaine at a concentration of 1% w/v; b) a PLGA-PEG-PLGA triblock copolymer component at a concentration of 20% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 2% v/v; and d) PEG 400 at a concentration of 15% v/v.

The present disclosure provides a composition of lidocaine formulated in a PLGA-PEG-PLGA triblock copolymer component, wherein the composition comprises: a) lidocaine at a concentration of 1% w/v; b) a PLGA-PEG-PLGA triblock copolymer component at a concentration of 17% w/v: (i) wherein the PLGA-PEG-PLGA triblock copolymer component comprises one 1700 Da-1500 Da-1700 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 2% v/v; and d) PEG 400 at a concentration of 15% v/v.

The present disclosure provides a composition of lidocaine formulated in a PLGA-PEG-PLGA triblock copolymer component, wherein the composition comprises: a) lidocaine at a concentration of 1% w/v; b) a PLGA-PEG-PLGA triblock copolymer component at a concentration of 23.5% w/v: (i) wherein the PLGA-PEG-PLGA triblock copolymer component comprises one 1700 Da-1500 Da-1700 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 1% v/v; and d) PEG 400 at a concentration of 5% v/v.

The present disclosure provides a composition of lidocaine formulated in a PLGA-PEG-PLGA triblock copolymer component, wherein the composition comprises: a) lidocaine at a concentration of 1% w/v; b) a PLGA-PEG-PLGA triblock copolymer component at a concentration of 26% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 75% of lactic acid (LA) and 25% of glycolic acid (GA) (LA:GA ratio of 75:25); and (v) wherein the triblock copolymer component comprises the first PLGA-PEG-PLGA triblock copolymer at a concentration of 54% of the triblock copolymer component and the second PLGA-PEG-PLGA triblock copolymer at a concentration of 46% of the triblock copolymer component; c) DMA at a concentration of 1% v/v; and d) PEG 400 at a concentration of 5% v/v.

The present disclosure provides a composition of lidocaine formulated in a PLGA-PEG-PLGA triblock copolymer component, wherein the composition comprises: a) lidocaine at a concentration of 1% w/v; b) a PLGA-PEG-PLGA triblock copolymer component at a concentration of 23.5% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1100 Da-1000 Da-1100 Da triblock copolymer and the second PLGA-PEG-PLGA triblock copolymer is a 1700 Da-1500 Da-1700 Da of PLGA-PEG-PLGA; (iii) wherein the first PLGA-PEG-PLGA triblock copolymer comprises 75% of lactic acid (LA) and 25% of glycolic acid (GA) (LA:GA ratio of 75:25); (iv) wherein the second PLGA-PEG-PLGA triblock copolymer comprises 100% of lactic acid (LA) and 0% of glycolic acid (GA) (LA:GA ratio of 100:0); and (v) wherein the triblock copolymer component comprises the first PLGA-PEG-PLGA triblock copolymer at a concentration of 50% of the triblock copolymer component and the second PLGA-PEG-PLGA triblock copolymer at a concentration of 40% of the triblock copolymer component; c) DMA at a concentration of 1% v/v; and d) PEG 400 at a concentration of 5% v/v.

The present disclosure provides a composition of bupivacaine formulated in a PLGA-PEG-PLGA triblock copolymer component, wherein the composition comprises: a) bupivacaine at a concentration of 1% w/v; b) a PLGA-PEG-PLGA triblock copolymer component at a concentration of 23% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 10% v/v; and d) PEG 400 at a concentration of 15% v/v.

The present disclosure provides a composition of bupivacaine formulated in a PLGA-PEG-PLGA triblock copolymer component, wherein the composition comprises: a) bupivacaine at a concentration of 0.5% w/v; b) a PLGA-PEG-PLGA triblock copolymer component at a concentration of 17% w/v: (i) wherein the PLGA-PEG-PLGA triblock copolymer component comprises one 1100 Da-1000 Da-1100 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 5% v/v; and d) PEG 400 at a concentration of 15% v/v.

The present disclosure provides a composition of bupivacaine formulated in a PLGA-PEG-PLGA triblock copolymer component, wherein the composition comprises: a) bupivacaine at a concentration of 0.5% w/v; b) a PLGA-PEG-PLGA triblock copolymer component at a concentration of 24% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 75% of lactic acid (LA) and 25% of glycolic acid (GA) (LA:GA ratio of 75:25); and (v) wherein the triblock copolymer component comprises the first PLGA-PEG-PLGA triblock copolymer at a concentration of 78% of the triblock copolymer component and the second PLGA-PEG-PLGA triblock copolymer at a concentration of 22% of the triblock copolymer component; c) DMA at a concentration of 5% v/v; and d) PEG 400 at a concentration of 10% v/v.

The present disclosure provides a composition of bupivacaine formulated in a PLGA-PEG-PLGA triblock copolymer component, wherein the composition comprises: a) bupivacaine at a concentration of 0.5% w/v; b) a PLGA-PEG-PLGA triblock copolymer component at a concentration of 20% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1700 Da-1500 Da-1700 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 100% of lactic acid (LA) and 0% of glycolic acid (GA) (LA:GA ratio of 100:0); and (v) wherein the triblock copolymer component comprises the first PLGA-PEG-PLGA triblock copolymer at a concentration of 60% of the triblock copolymer component and the second PLGA-PEG-PLGA triblock copolymer at a concentration of 40% of the triblock copolymer component; c) DMA at a concentration of 5% v/v; and d) PEG 400 at a concentration of 15% v/v.

The present disclosure provides a composition of ropivacaine formulated in a PLGA-PEG-PLGA triblock copolymer component, wherein the composition comprises: a) ropivacaine at a concentration of 0.4% w/v; b) a PLGA-PEG-PLGA triblock copolymer component at a concentration of 23% w/v: (i) wherein the PLGA-PEG-PLGA triblock copolymer component comprises one 1400 Da-1500 Da-1400 Da of PLGA-PEG-PLGA triblock copolymer, and (ii) wherein the PLGA comprises 50% of lactic acid (LA) and 50% of glycolic acid (GA) (LA:GA ratio of 50:50); c) DMA at a concentration of 10% v/v; and d) PEG 400 at a concentration of 15% v/v.

The present disclosure provides a composition of ropivacaine formulated in a PLGA-PEG-PLGA triblock copolymer component, wherein the composition comprises: a) ropivacaine at a concentration of 0.4% w/v; b) a PLGA-PEG-PLGA triblock copolymer component at a concentration of 17% w/v: (i) wherein the PLGA-PEG-PLGA triblock copolymer component comprises one 1100 Da-1000 Da-1100 Da of PLGA-PEG-PLGA triblock copolymer, and (ii) wherein the PLGA comprises 100% of lactic acid (LA) and 0% of glycolic acid (GA) (LA:GA ratio of 100:0); c) DMA at a concentration of 5% v/v; and d) PEG 400 at a concentration of 15% v/v.

The present disclosure provides a composition of ropivacaine formulated in a PLGA-PEG-PLGA triblock copolymer component, wherein the composition comprises: a) ropivacaine at a concentration of 0.4% w/v; b) a PLGA-PEG-PLGA triblock copolymer component at a concentration of 24% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 75% of lactic acid (LA) and 25% of glycolic acid (GA) (LA:GA ratio of 75:25); and (v) wherein the triblock copolymer component comprises the first PLGA-PEG-PLGA triblock copolymer at a concentration of 78% of the triblock copolymer component and the second PLGA-PEG-PLGA triblock copolymer at a concentration of 22% of the triblock copolymer component; c) DMA at a concentration of 5% v/v; and d) PEG 400 at a concentration of 10% v/v.

The present disclosure provides a composition of ropivacaine formulated in a PLGA-PEG-PLGA triblock copolymer component, wherein the composition comprises: a) ropivacaine at a concentration of 0.4% w/v; b) a PLGA-PEG-PLGA triblock copolymer component at a concentration of 20% w/v: (i) wherein the PLGA-PEG-PLGA triblock copolymer component comprises a first and a second PLGA-PEG-PLGA triblock copolymers; (ii) wherein the first PLGA-PEG-PLGA triblock copolymer is a 1400 Da-1500 Da-1400 Da triblock copolymer and the second PLGA-PEG-PLGA triblock copolymer is a 1700 Da-1500 Da-1700 Da of PLGA-PEG-PLGA; (iii) wherein the first PLGA-PEG-PLGA triblock copolymer comprises 50% of lactic acid (LA) and 50% of glycolic acid (GA) (LA:GA ratio of 50:50); (iv) wherein the second PLGA-PEG-PLGA triblock copolymer comprises 100% of lactic acid (LA) and 0% of glycolic acid (GA) (LA:GA ratio of 100:0); and (v) wherein the triblock copolymer component comprises the first PLGA-PEG-PLGA triblock copolymer at a concentration of 60% of the triblock copolymer component and the second PLGA-PEG-PLGA triblock copolymer at a concentration of 40% of the triblock copolymer component; c) DMA at a concentration of 5% v/v; and d) PEG 400 at a concentration of 15% v/v.

The molecular weights of the polymer components, PLGA and PEG of the triblock polymer of the composition as disclosed herein can be reported as weight average (Mw) and Number average (Mn). For consistency and clarity, Molecular Weights reported herein are reported as Mn as measured by Gel Permeation Chromatography (GPC). Polymer molecular weight, when measured by GPC can vary substantially based on method and system differences, including instrument manufacturers, column types and manufacturers, reagents, lab environments and standards used.

The composition can have a dose volume of 0.5 ml to 50 ml (or any integer therein). The composition can have a dose volume of 1 ml to 50 ml (or any integer therein). The composition can have a dose volume of 5 ml to 40 ml (or any integer therein). The composition can have a dose volume of 5 ml to 10 ml (or any integer therein). The composition can have a dose volume of 10 ml to 20 ml (or any integer therein). The composition can have a dose volume of 20 ml to 30 ml (or any integer therein). The composition can have a dose volume of 30 ml to 40 ml (or any integer therein). The total dose volume of the composition can be 0.5 ml. The total dose volume of the composition can be 1 ml. The total dose volume of the composition can be 5 ml. The total dose volume of the composition can be 10 ml. The total dose volume of the composition can be 15 ml. The total dose volume of the composition can be 20 ml. The total dose volume of the composition can be 25 ml. The total dose volume of the composition can be 30 ml. The total dose volume of the composition can be 35 ml. The total dose volume of the composition can be 40 ml.

The present disclosure provides a composition of lidocaine formulated in a triblock copolymer component that comprises one PDLLA-PEG-PDLLA or PLA-PEG-PLA triblock copolymer, which is the same as PLGA-PEG-PLGA where the PLGA comprises 100% of lactic acid (LA) and 0% of glycolic acid (GA) (LA:GA ratio of 100:0). The present disclosure provides a composition of lidocaine formulated in a triblock copolymer component that comprises one PLGA-PEG-PLGA where the PLGA comprises 75% of lactic acid (LA) and 25% of glycolic acid (GA) (LA:GA ratio of 75:25). The present disclosure provides a composition of lidocaine formulated in a triblock copolymer component that comprises one PLGA-PEG-PLGA where the PLGA comprises 50% of lactic acid (LA) and 50% of glycolic acid (GA) (LA:GA ratio of 50:50).

The present disclosure provides a composition of lidocaine formulated in a triblock copolymer component that comprises a first PLGA-PEG-PLGA where the PLGA comprises 100% of lactic acid (LA) and 0% of glycolic acid (GA) (LA:GA ratio of 100:0), and a second PLGA-PEG-PLGA where the PLGA comprises 75% of lactic acid (LA) and 25% of glycolic acid (GA) (LA:GA ratio of 75:25). The present disclosure provides a composition of lidocaine formulated in a triblock copolymer component that comprises a first PLGA-PEG-PLGA where the PLGA comprises 100% of lactic acid (LA) and 0% of glycolic acid (GA) (LA:GA ratio of 100:0), and a second PLGA-PEG-PLGA where the PLGA comprises 50% of lactic acid (LA) and 50% of glycolic acid (GA) (LA:GA ratio of 50:50). The present disclosure provides a composition of lidocaine formulated in a triblock copolymer component that comprises a first PLGA-PEG-PLGA where the PLGA comprises 75% of lactic acid (LA) and 25% of glycolic acid (GA) (LA:GA ratio of 75:25), and a second PLGA-PEG-PLGA where the PLGA comprises 50% of lactic acid (LA) and 50% of glycolic acid (GA) (LA:GA ratio of 50:50).

The present disclosure provides a composition of bupivacaine formulated in a triblock copolymer component that comprises one PDLLA-PEG-PDLLA or PLA-PEG-PLA triblock copolymer, which is the same as PLGA-PEG-PLGA where the PLGA comprises 100% of lactic acid (LA) and 0% of glycolic acid (GA) (LA:GA ratio of 100:0). The present disclosure provides a composition of bupivacaine formulated in a triblock copolymer component that comprises one PLGA-PEG-PLGA where the PLGA comprises 75% of lactic acid (LA) and 25% of glycolic acid (GA) (LA:GA ratio of 75:25). The present disclosure provides a composition of bupivacaine formulated in a triblock copolymer component that comprises one PLGA-PEG-PLGA where the PLGA comprises 50% of lactic acid (LA) and 50% of glycolic acid (GA) (LA:GA ratio of 50:50).

The present disclosure provides a composition of bupivacaine formulated in a triblock copolymer component that comprises a first PLGA-PEG-PLGA where the PLGA comprises 100% of lactic acid (LA) and 0% of glycolic acid (GA) (LA:GA ratio of 100:0), and a second PLGA-PEG-PLGA where the PLGA comprises 75% of lactic acid (LA) and 25% of glycolic acid (GA) (LA:GA ratio of 75:25). The present disclosure provides a composition of bupivacaine formulated in a triblock copolymer component that comprises a first PLGA-PEG-PLGA where the PLGA comprises 100% of lactic acid (LA) and 0% of glycolic acid (GA) (LA:GA ratio of 100:0), and a second PLGA-PEG-PLGA where the PLGA comprises 50% of lactic acid (LA) and 50% of glycolic acid (GA) (LA:GA ratio of 50:50). The present disclosure provides a composition of bupivacaine formulated in a triblock copolymer component that comprises a first PLGA-PEG-PLGA where the PLGA comprises 75% of lactic acid (LA) and 25% of glycolic acid (GA) (LA:GA ratio of 75:25), and a second PLGA-PEG-PLGA where the PLGA comprises 50% of lactic acid (LA) and 50% of glycolic acid (GA) (LA:GA ratio of 50:50).

The present disclosure provides a composition of ropivacaine formulated in a triblock copolymer component that comprises one PDLLA-PEG-PDLLA or PLA-PEG-PLA triblock copolymer, which is the same as PLGA-PEG-PLGA where the PLGA comprises 100% of lactic acid (LA) and 0% of glycolic acid (GA) (LA:GA ratio of 100:0). The present disclosure provides a composition of ropivacaine formulated in a triblock copolymer component that comprises one PLGA-PEG-PLGA where the PLGA comprises 75% of lactic acid (LA) and 25% of glycolic acid (GA) (LA:GA ratio of 75:25). The present disclosure provides a composition of ropivacaine formulated in a triblock copolymer component that comprises one PLGA-PEG-PLGA where the PLGA comprises 50% of lactic acid (LA) and 50% of glycolic acid (GA) (LA:GA ratio of 50:50).

The present disclosure provides a composition of ropivacaine formulated in a triblock copolymer component that comprises a first PLGA-PEG-PLGA where the PLGA comprises 100% of lactic acid (LA) and 0% of glycolic acid (GA) (LA:GA ratio of 100:0), and a second PLGA-PEG-PLGA where the PLGA comprises 75% of lactic acid (LA) and 25% of glycolic acid (GA) (LA:GA ratio of 75:25). The present disclosure provides a composition of ropivacaine formulated in a triblock copolymer component that comprises a first PLGA-PEG-PLGA where the PLGA comprises 100% of lactic acid (LA) and 0% of glycolic acid (GA) (LA:GA ratio of 100:0), and a second PLGA-PEG-PLGA where the PLGA comprises 50% of lactic acid (LA) and 50% of glycolic acid (GA) (LA:GA ratio of 50:50). The present disclosure provides a composition of ropivacaine formulated in a triblock copolymer component that comprises a first PLGA-PEG-PLGA where the PLGA comprises 75% of lactic acid (LA) and 25% of glycolic acid (GA) (LA:GA ratio of 75:25), and a second PLGA-PEG-PLGA where the PLGA comprises 50% of lactic acid (LA) and 50% of glycolic acid (GA) (LA:GA ratio of 50:50).

The present disclosure provides a method of treatment for post-surgical pain in a subject in need thereof, wherein the method comprises administering an effective amount of any of the compositions of the disclosure. The method of treatment for post-surgical pain in a subject in need thereof can comprise administering an effective amount of any of the compositions of the disclosure to the subject prior to a surgical procedure. The method of treatment for post-surgical pain in a subject in need thereof can comprise administering an effective amount of any of the compositions of the disclosure to the subject during a surgical procedure. The method of treatment for post-surgical pain in a subject in need thereof can comprise administering an effective amount of any of the compositions of the disclosure to the subject following a surgical procedure. The method of treatment for post-surgical pain in a subject in need thereof can comprise administering an effective amount of any of the compositions of the disclosure to the subject, prior to, during and following a surgical procedure.

The present disclosure provides a method of prevention or treatment for pain during a surgical procedure in a subject in need thereof, wherein the method comprises administering an effective amount of the composition of the disclosure. The method of prevention or treatment for pain during a surgical procedure in a subject in need thereof can comprise administering an effective amount of any of the compositions of the disclosure to the subject prior to a surgical procedure. The method of prevention or treatment for pain during a surgical procedure in a subject in need thereof can comprise administering an effective amount of any of the compositions of the disclosure to the subject during a surgical procedure. The method of prevention or treatment for pain during a surgical procedure in a subject in need thereof can comprise administering an effective amount of any of the compositions of the disclosure to the subject prior to and during a surgical procedure.

The method of treatment can comprise administering an effective amount of any of the compositions of the disclosure to the subject 1 minute to 8 hours prior to a surgical procedure. The method of treatment can comprise administering an effective amount of any of the compositions of the disclosure to the subject 15 minutes to 8 hours prior to a surgical procedure. The method of treatment can comprise administering an effective amount of any of the compositions of the disclosure to the subject 30 minutes to 8 hours prior to a surgical procedure. The method of treatment can comprise administering an effective amount of any of the composition of the disclosure to the subject 1 to 8 hours prior to a surgical procedure. The method of treatment can comprise administering an effective amount of any of the compositions of the disclosure to the subject 1 to 6 hours prior to a surgical procedure. The method of treatment can comprise administering an effective amount of any of the compositions of the disclosure to the subject 1 to 3 hours prior to a surgical procedure. The method of treatment can comprise administering an effective amount of any of the compositions of the disclosure to the subject 1 to 4 hours prior to a surgical procedure. The method of treatment can comprise administering an effective amount of any of the compositions of the disclosure to the subject 1 to 2 hours prior to a surgical procedure. The method of treatment can comprise administering an effective amount of any of the compositions of the disclosure to the subject 2 to 4 hours prior to a surgical procedure. The method of treatment can comprise administering an effective amount of any of the compositions of the disclosure to the subject 4 to 6 hours prior to a surgical procedure. The method of treatment can comprise administering an effective amount of any of the compositions of the disclosure to the subject 6 to 8 hours prior to a surgical procedure. The method of treatment can comprise administering an effective amount of any of the compositions of the disclosure to the subject 1 minutes, 15 minutes, 30 minutes or 1, 2, 3, 4, 5, 6, 7 or 8 hours prior to a surgical procedure.

The method of treatment can comprise administering an effective amount of any of the compositions of the disclosure to the subject 1 minute-240 hours following a surgical procedure. The method of treatment can comprise administering an effective amount of any of the compositions of the disclosure to the subject 15 minutes-240 hours following a surgical procedure. The method of treatment can comprise administering an effective amount of any of the compositions of the disclosure to the subject 30 minutes-240 hours following a surgical procedure. The method of treatment can comprise administering an effective amount of any of the compositions of the disclosure to the subject 1-240 hours following a surgical procedure. The method of treatment can comprise administering an effective amount of any of the compositions of the disclosure to the subject 1 to 8 hours following a surgical procedure. The method of treatment can comprise administering an effective amount of any of the compositions of the disclosure to the subject 1 to 6 hours following a surgical procedure. The method of treatment can comprise administering an effective amount of any of the compositions of the disclosure to the subject 1 to 3 hours following a surgical procedure. The method of treatment can comprise administering an effective amount of any of the compositions of the disclosure to the subject 1 to 4 hours following a surgical procedure. The method of treatment can comprise administering an effective amount of any of the compositions of the disclosure to the subject 1 to 2 hours following a surgical procedure. The method of treatment can comprise administering an effective amount of any of the compositions of the disclosure to the subject 2 to 4 hours following a surgical procedure. The method of treatment can comprise administering an effective amount of any of the compositions of the disclosure to the subject 4 to 6 hours following a surgical procedure. The method of treatment can comprise administering an effective amount of any of the compositions of the disclosure to the subject 6 to 8 hours following a surgical procedure. The method of treatment can comprise administering an effective amount of any of the compositions of the disclosure to the subject 8 to 10 hours following a surgical procedure. The method of treatment can comprise administering an effective amount of any of the compositions of the disclosure to the subject 10 to 12 hours following a surgical procedure. The method of treatment can comprise administering an effective amount of any of the compositions of the disclosure to the subject 12 to 24 hours following a surgical procedure. The method of treatment can comprise administering an effective amount of any of the compositions of the disclosure to the subject 24 to 48 hours following a surgical procedure. The method of treatment can comprise administering an effective amount of any of the compositions of the disclosure to the subject 48 to 72 hours following a surgical procedure. The method of treatment can comprise administering an effective amount of any of the compositions of the disclosure to the subject 72 to 96 hours following a surgical procedure. The method of treatment can comprise administering an effective amount of any of the compositions of the disclosure to the subject 96 to 120 hours following a surgical procedure. The method of treatment can comprise administering an effective amount of any of the compositions of the disclosure to the subject 120 to 144 hours following a surgical procedure. The method of treatment can comprise administering an effective amount of any of the compositions of the disclosure to the subject 144 to 168 hours following a surgical procedure. The method of treatment can comprise administering an effective amount of any of the compositions of the disclosure to the subject 168 to 192 hours following a surgical procedure. The method of treatment can comprise administering an effective amount of any of the compositions of the disclosure to the subject 192 to 216 hours following a surgical procedure. The method of treatment can comprise administering an effective amount of any of the compositions of the disclosure to the subject 216 to 240 hours following a surgical procedure. The method of treatment can comprise administering an effective amount of any of the compositions of the disclosure to the subject 1 minute, 15 minutes, 30 minutes or 1, 2, 3, 4, 5, 6, 7, 8, 9, 10, 12, 24, 48, 72, 96, 120, 144, 168, 192, 216 or 240 hours following a surgical procedure.

The method of treatment can comprise administering an effective amount of any of the compositions of the disclosure to the subject prior to a surgical procedure and an effective amount of any of the compositions of the disclosure to the subject following a surgical procedure. The method of treatment can comprise administering an effective amount of any of the compositions of the disclosure to the subject 1 minute to 1 hour or 1 to 8 hours prior to a surgical procedure, and administering an effective amount of any of the compositions of the disclosure to the subject 1 minute to 1 hour or 1 to 240 hours following the surgical procedure.

The method of treatment can comprise administering an effective amount of any of the compositions of the disclosure to the subject 1 to 6 hours prior to a surgical procedure, and administering an effective amount of any of the compositions of the disclosure to the subject 1 to 240 hours following the surgical procedure. The method of treatment can comprise administering an effective amount of any of the compositions of the disclosure to the subject 1 to 3 hours prior to a surgical procedure, and administering an effective amount any of the compositions of the disclosure to the subject 1 to 240 hours following the surgical procedure. The method of treatment can comprise administering an effective amount of any of the compositions of the disclosure to the subject 1 to 4 hours prior to a surgical procedure, and administering an effective amount of any of the compositions of the disclosure to the subject 1 to 240 hours following the surgical procedure. The method of treatment can comprise administering an effective amount of any of the compositions of the disclosure to the subject 1 to 2 hours prior to a surgical procedure, and administering an effective amount of any of the compositions of the disclosure to the subject 1 to 240 hours following the surgical procedure. The method of treatment can comprise administering an effective amount of any of the compositions of the disclosure to the subject 2 to 4 hours prior to a surgical procedure, and administering an effective amount of any of the compositions of the disclosure to the subject 1 to 240 hours following the surgical procedure. The method of treatment can comprise administering an effective amount of any of the compositions of the disclosure to the subject 4 to 6 hours prior to a surgical procedure, and administering an effective amount of any of the compositions of the disclosure to the subject 1 to 240 hours following the surgical procedure. The method of treatment can comprise administering an effective amount of any of the compositions of the disclosure to the subject 6 to 8 hours prior to a surgical procedure, and administering an effective amount of any of the compositions of the disclosure to the subject 1 to 240 hours following the surgical procedure. The method of treatment can comprise administering an effective amount of any of the compositions of the disclosure to the subject 1 minute, 15 minutes, 30 minutes or 1, 3, 3, 4, 5, 6, 7 or 8 hours prior to a surgical procedure, and administering an effective amount of any of the compositions of the disclosure to the subject 1 minute, 15 minutes, 30 minutes or 1 to 240 hours following the surgical procedure.

The method of treatment can comprise administering an effective amount of any of the compositions of the disclosure to the subject 1 minute to 1 hour or 1 to 8 hours prior to a surgical procedure, and administering an effective amount of any of the compositions of the disclosure to the subject 1 minute to 1 hour or 1 to 240 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 1 to 8 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 1 to 6 hours following a surgical procedure following the surgical procedure. The method of treatment can comprise administering an effective amount of any of the compositions of the disclosure to the subject 1 to 8 hours prior to a surgical procedure, and administering an effective amount of any of the compositions of the disclosure to the subject 1 to 4 hours following a surgical procedure following the surgical procedure. The method of treatment can comprise administering an effective amount of any of the compositions of the disclosure to the subject 1 to 8 hours prior to a surgical procedure, and administering an effective amount of any of the compositions of the disclosure to the subject 1 to 3 hours following a surgical procedure following the surgical procedure. The method of treatment can comprise administering an effective amount of any of the compositions of the disclosure to the subject 1 to 8 hours prior to a surgical procedure, and administering an effective amount of any of the compositions of the disclosure to the subject 1 to 2 hours following a surgical procedure following the surgical procedure.

The method of treatment can comprise administering an effective amount of any of the compositions of the disclosure to the subject 1 minute to 1 hour or 1 to 8 hours prior to a surgical procedure, and administering an effective amount of any of the compositions of the disclosure to the subject 1 minute to 2 hours or 2 to 4 hours following a surgical procedure following the surgical procedure. The method of treatment can comprise administering an effective amount of any of the compositions of the disclosure to the subject 1 to 8 hours prior to a surgical procedure, and administering an effective amount of any of the compositions of the disclosure to the subject 4 to 6 hours following a surgical procedure following the surgical procedure. The method of treatment can comprise administering an effective amount of any of the compositions of the disclosure to the subject 1 to 8 hours prior to a surgical procedure, and administering an effective amount of any of the compositions of the disclosure to the subject 6 to 8 hours following a surgical procedure following the surgical procedure. The method of treatment can comprise administering an effective amount of any of the compositions of the disclosure to the subject 1 to 8 hours prior to a surgical procedure, and administering an effective amount of any of the compositions of the disclosure to the subject 8 to 10 hours following a surgical procedure following the surgical procedure. The method of treatment can comprise administering an effective amount of any of the compositions of the disclosure to the subject 1 to 8 hours prior to a surgical procedure, and administering an effective amount of any of the compositions of the disclosure to the subject 10 to 12 hours following a surgical procedure following the surgical procedure. The method of treatment can comprise administering an effective amount of any of the compositions of the disclosure to the subject 1 to 8 hours prior to a surgical procedure, and administering an effective amount of any of the compositions of the disclosure to the subject 12 to 24 hours following a surgical procedure following the surgical procedure.

The method of treatment can comprise administering an effective amount of any of the compositions of the disclosure to the subject 1 to 8 hours prior to a surgical procedure, and administering an effective amount of any of the compositions of the disclosure to the subject 24 to 48 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 48 to 72 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 71 to 96 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 96 to 120 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 122 to 144 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 144 to 168 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 168 to 192 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 192 to 216 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 216 to 240 hours following the surgical procedure. The method of treatment can comprise administering an effective amount of any of the compositions of the disclosure to the subject 1 to 8 hours prior to a surgical procedure, and administering an effective amount of any of the compositions of the disclosure to the subject 1 minute, 15 minutes, 30 minutes or 1, 2, 3, 4, 5, 6, 7, 8, 9, 10, 12, 24, 48, 72, 96, 120, 144, 168, 192, 216 or 240 hours following the surgical procedure.

The present disclosure provides a method of treatment for pain in a subject in need thereof, wherein the method comprises administering to the subject an effective amount of any of the compositions disclosed herein. The pain can be acute pain. The acute pain can be due to any one of an injury, dental work or labor and childbirth, or any combination thereof. The term “acute pain” is used herein in its broadest sense to refer to pain that has a duration of less than 6 months, preferably less than 3 months. As would be appreciated by the skilled artisan, acute pain can be attributed to a specific cause (e.g. injury, dental work, labor and childbirth, etc.) and typically subsides after the specific cause is treated.

The method of treatment can comprise administering an effective amount of any of the compositions of the disclosure to the subject wherein the effective amount induces an analgesic nerve block. The effective amount induces an analgesic nerve block prior to surgical procedure. The effective amount induces an analgesic nerve block during a surgical procedure. The effective amount induces an analgesic nerve block following a surgical procedure. The effective amount induces an analgesic nerve block following an injury. The effective amount induces an analgesic nerve block following a surgical procedure. The effective amount induces an analgesic nerve block prior to dental work. The effective amount induces an analgesic nerve block during dental work. The effective amount induces an analgesic nerve block during labor and childbirth, including Cesarean delivery if required.

The analgesic nerve block can be any one of upper extremity nerve block, lower extremity nerve block, facial nerve block, neck and back nerve block, chest and abdominal block, or any combination thereof. The upper extremity nerve block can be any one of interscalene, supraclavicular, brachial plexus and infraclavicular nerve block, or any combination thereof. The lower extremity nerve block can be any one of hypogastric plexus, lumbar plexus, femoral nerve, adductor canal nerves, popliteal fossa and sciatic nerve, or any combination thereof. The facial nerve block can be any one of trigeminal, ophthalmic, supraorbital, maxillary, sphenopalatine, or any combination thereof. The neck and back nerve block can be any one of cervical epidural, thoracic epidural, lumbar epidural, caudal block or any combination thereof. The chest and abdominal block can be any one of paravertebral, intercostal and transversus abdominis plane, rectus sheath or any combination thereof.

The surgical procedure can be any one of diagnostic surgical procedure, ablative surgical procedure, palliative surgical procedure, reconstructive surgical procedure, transplantation surgical procedure, constructive surgical procedure, or any combination thereof.

The surgical procedure can be any one of upper extremity surgical procedure, lower extremity surgical procedure, abdominal surgical procedure, cardio-thoracic surgical procedure, head and neck surgical procedure, back surgical procedure, musculoskeletal surgical procedure, orthopedic surgical procedure, ocular surgical procedure, ear, nose and throat surgical procedure, vascular surgical procedure, dental surgical procedure, or any combination thereof.

The surgical procedure can be any one of upper extremity surgical procedure and lower extremity surgical procedure, or any combination thereof. The upper extremity surgical procedure can be any one of a surgical procedure of arm, forearm, hand, wrist, elbow, shoulder, or any combination thereof. The upper extremity surgical procedure can be a surgical procedure of one or more bone(s) of the arm, forearm, hand, wrist, elbow, shoulder, or any combination thereof. The one or more bone(s) can comprise any one of radius, ulna, humerus, carpals, metacarpals or phalanges or a combination thereof.

The elbow and shoulder surgical procedure can be any one of a fracture surgery, total shoulder replacement, reverse shoulder replacement, shoulder arthroscopy, elbow arthroscopy, rotator cuff repair, surgery for tennis and golfer's elbow, or any combination thereof. The hand and wrist surgical procedure can be any one of a fracture surgery, carpal tunnel release, wrist arthroscopy, wrist joint replacement, wrist fusion, or any combination thereof.

The lower extremity surgical procedure is any one of a surgical procedure of hip, thigh, knee, ankle, feet, or any combination thereof. The lower extremity surgical procedure can be a surgical procedure of one or more bone(s) of the hip, thigh, knee, ankle, feet, or any combination thereof. The one or more bone(s) can comprise any one of acetabulum, femur, tibia, fibula, patella, tarsal bone, metatarsal bone, arches of the foot or a combination thereof.

The lower extremity surgical procedure can be any one of arthroscopy (diagnostic tool and treatment method to see inside joints), bone fusion, open-reduction fracture setting, surgical removal (i.e. of bunions, cysts, nail, etc.), tendon and ligament repair or any combination thereof.

The abdominal surgical procedure can be any one of a surgical procedure of the stomach, small intestines, spleen, appendix and the colon (or rectum). The abdominal surgical procedure can be any one of appendectomy, cholecystectomy, colon/anorectal surgery, colon/large bowel resections, hernia surgery, small bowel resection, or any combination thereof.

The cardio-thoracic surgical procedure can be any one of a surgical procedure of the lung or heart. The cardio-thoracic surgical procedure can be any one of a surgical procedure of the respiratory system, the cardiovascular system, or any combination thereof. The cardio-thoracic surgical procedure can be any one of thoracotomy, lobectomy, pneumonectomy, lung transplant, surgery to prevent pleurodesis, surgery to prevent empyema, surgery to remove blood in the chest cavity (particularly after trauma heart transplant), surgery to remove small balloon-like tissues (blebs) that cause lung collapse (pneumothorax), wedge resection, aortic surgery, atrial fibrillation surgery (hybrid-maze procedure), carotid endarterectomy, coronary artery bypass surgery (CABG), heart valve repair/replacement surgery, heart valve replacement without surgery, mitral valve repair, minimally invasive cardiac surgery, robotic thoracic surgery, or any combination thereof.

The head and neck surgical procedure can be any one of a surgery for pinched nerves, intense dizziness and motion sickness, pressure on spinal cord, upper spinal issues, head and neck injuries, sinus pain, chronic ear infections, snoring and sleep apnea, tonsil issues, jaw issues and deformities, thyroid conditions, or any combination thereof.

The back surgical procedure can be a surgical procedure for correcting or repairing defects or injuries of the vertebral column. The back surgical procedures can be any one of a surgical procedure for correction or repair of herniated or ruptured disks, spinal stenosis, spondylolisthesis, vertebral fractures, degenerative disk disease, or any combination thereof.

The orthopedic surgical procedure can be any one of arthroscopy, soft tissue repair, joint replacement, revision joint surgery, bone fracture repair, debridement, fusion of bones, spine fusion, osteotomy, or any combination thereof. The orthopedic surgical procedure can be any one of knee arthroscopy and meniscectomy, shoulder arthroscopy and decompression, carpal tunnel release, knee arthroscopy and chondroplasty, removal of support implant, knee arthroscopy and anterior cruciate ligament reconstruction, knee replacement, repair of femoral neck fracture, repair of trochanteric fracture, hip replacement, or any combination thereof.

The ocular surgical procedure can be any one of oculoplastic surgery, surgical procedure of cornea, retina and eye muscle, refractive surgery, glaucoma surgery, or any combination thereof.

The vascular surgical procedure can be any one of aortic aneurysm repair aortocaval fistula repair, aortoenteric fistula repair, arteriovenous fistula surgery, arteriovenous malformation surgery, bypass surgery, carotid angioplasty and stenting, carotid artery stenting, carotid endarterectomy, celiac artery bypass, coronary angioplasty and stents, endovascular reconstruction, endovenous laser therapy, ex vivo renal artery reconstructions, inferior vena cava (ivc) filter retrieval, inferior vena cava (ivc) placement, laser ablation, mesenteric artery bypass, open vascular reconstruction, radiofrequency ablation, renal artery angioplasty, renal artery bypass, renal artery endarterectomy, stenting to repair aneurysms, subfascial endoscopic perforator surgery, thoracic duct embolization, thoracic outlet decompression, thrombectomy, vascular stenting, vein removal, vena cava reconstructions, vertebral artery reconstructions, or any combination thereof.

The dental surgical procedure can be any one of endodontics, root canal surgery, apicoectomy, prosthodontic surgery, orthodontic surgery, periodontic surgery, oral and maxillofacial surgery, or any combination thereof.

The surgical procedure can be of any one of an abdominal wall, a thoracic wall or a combination thereof. The surgical procedure of the abdominal wall can be a surgical procedure for abdominal wall reconstruction and repair. The surgical procedure of the abdominal wall can be a surgical procedure for abdominal wall hernia.

The surgical procedure can be a surgical procedure of an organ. The organ can be any one of heart, lung, liver, spleen, brain, stomach, small intestine, large intestine, gall bladder, bile ducts, lymphatic ducts, urinary bladder, prostate gland, pancreas, adrenal gland, thyroid gland, skin, uterus, fallopian tubes, ovary, seminal tubules, testes, or any combination thereof. The surgical procedure can be for any one of repair, removal or transplantation of the organ. The organ surgical procedure can be for removal of tumor or cancerous tissue from within or near the organ.

The surgical procedure can be any one of upper extremity surgical procedure and lower extremity surgical procedure. The surgical procedure can be any one of bunionectomy, total knee arthroplasty, anterior cruciate ligament reconstruction, shoulder arthroplasty (total or reverse), rotator cuff repair or a combination thereof.

The subject in need thereof, can be a subject that is to undergo, is undergoing or has undergone a surgical procedure can be a human. The subject can be a mammal. The subject can be a human, a murine, an equine, a canine, a feline, a rabbit, a pig, a hamster, a bovine or a camelid. The subject can be a human.

The effective amount of the composition can be administered perineurally, epidurally, subcutaneously, intradermally, orally, intramuscularly or intravenously. The effective amount of the formulation can be administered perineurally. The effective amount of the formulation is administered by direct instillation into a wound near the site of surgical procedure. The effective amount of the formulation is administered by the effective amount of the formulation is administered by infiltration at the site of surgical procedure.

Dosage

The disclosure provides an effective amount of the composition of the disclosure, for use in a method for managing pain in a subject in need thereof, caused by an injury, wound or surgical procedure while not materially impairing motor function. Managing pain can be defined as any one or all of: a) reducing pain intensity, and b) preventing need to or increasing the time to administer rescue anti-pain medication post-administration of the composition.

Managing pain can be defined as reducing the pain intensity in a subject that has undergone injury, wound or surgical procedure as measured on a numerical rating scale (NRS) scale. A NRS scale for assessing pain intensity of the subject in need thereof, on a defined 11-point (0-10), 21-point (0-20) and 101-point (0-100) scale (Jensen & Karoly 2001), wherein a score of 0 means “no pain”, scores of 10, 20 and 100 mean “worst pain imaginable, and scores between 0 and the highest point on the scale being mild to moderate pain in ascending order. The effective amount is an amount that causes reduction of pain intensity in the subject in need thereof, as measured on a NRS scale, from a score that means “worst pain imaginable” on Day 0 post-surgery/injury, to a score that means “no pain” at a time between 24 hours to 7 days post-surgery/injury. The effective amount is an amount that causes reduction of pain intensity in the subject in need thereof, as measured on a NRS scale, from a score that means “worst pain imaginable” at Day 0 post-surgery/injury, to a score that means “mild pain” at a time between 24 hours to 7 days post-surgery/injury. The effective amount is an amount that causes reduction of pain intensity in the subject in need thereof, as measured on a NRS scale, from a score that means “worst pain imaginable” at Day 0 post-surgery/injury, to a score that means “moderate pain” at a time between 24 hours to 7 days post-surgery/injury. The effective amount is an amount that causes reduction of pain intensity in the subject in need thereof, as measured on a NRS scale, from a score that means “moderate pain” at Day 0 post-surgery/injury, to a score that means “mild pain” at a time between 24 hours to 7 days post-surgery/injury.

The “effective amount” or “therapeutically effective amount” of the composition as disclosed herein, is an amount of the composition that upon administering to a subject in need thereof, reduces or inhibits pain or makes pain manageable in the subject. The “effective amount” or “therapeutically effective amount” of the composition as disclosed herein, is an amount of the composition, that causes reduction of pain intensity in the subject in need thereof, as measured on a 11-point NRS scale, from a score of 10 at Day 0 post-surgery/injury, to a score of 0 at a time between 24 hours to 7 days post-surgery/injury. The effective amount is an amount that causes reduction of pain intensity in the subject in need thereof, as measured on a 11-point NRS scale, from a score of 10 at Day 0 post-surgery/injury, to a score of 0-5 at a time between 24 hours to 7 days post-surgery/injury. The effective amount is an amount that causes reduction of pain intensity in the subject in need thereof, as measured on a 11-point NRS scale, from a score of 8-10 at Day 0 post-surgery/injury, to a score of 5-7 at a time between 24 hours to 7 days post-surgery/injury. The effective amount is an amount that causes reduction of pain intensity in the subject in need thereof, as measured on a 11-point NRS scale, from a score of 6-10 at Day 0 post-surgery/injury, to a score of 0-5 at a time between 24 hours to 7 days post-surgery/injury.

The loss of motor function in the subject that has undergone injury, wound or surgical procedure, can be measured by assessing motor strength, specifically, strength of knee extension and flexion, ankle dorsiflexion, and ankle plantar flexion.

The preventing the need to, or increasing the time to administer rescue anti-pain medication post-administration of the composition, to the subject that has undergone injury, wound or surgical procedure, can be assessed by determining the time post-surgery/injury, for which the subject can remain without concomitant use of an anti-pain medication. The anti-pain medication can be an opioid medication. The opioid medication can be any one of or a combination of oxycodone, morphine, codeine, fentanyl, hydrocodone, meperidine and methadone. The effective amount of the composition as disclosed herein can be an amount that prevents the need for administering an opioid medication less than 1 week post surgery/injury. The effective amount of the composition as disclosed herein can be an amount that prevents the need for administering an opioid medication less than 4 weeks post surgery/injury. An effective amount of the composition as disclosed herein can be an amount that prevents the need for administering an opioid medication less than 8 weeks post surgery/injury.

The method of treatment can comprise administering an effective amount of any of the compositions of the disclosure to the subject in need thereof, wherein the effective amount of the composition can be 1-20 mg/ml. The method of treatment can comprise administering an effective amount of any of the compositions of the disclosure to the subject in need thereof, wherein the effective amount of the composition can be 1.5-20 mg/ml. The method of treatment can comprise administering an effective amount of any of the compositions of the disclosure to the subject in need thereof, wherein the effective amount of the any of the compositions can be 2-20 mg/ml. The effective amount of the any of the compositions can be 6-13 mg/ml. The effective amount of the composition can be 2-5 mg/ml. The effective amount of the any of the compositions can be 5-10 mg/ml. The effective amount of the any of the compositions can be 10-15 mg/ml. The effective amount of the any of the compositions can be 15-20 mg/ml. The effective amount of the any of the compositions can be 3-6 mg/ml. The effective amount of the any of the compositions can be 6-13 mg/ml. The effective amount of the any of the compositions can be 6.5-13 mg/ml. The effective amount of the any of the compositions can be 10-13 mg/ml. The effective amount of the any of the compositions can be 13-16 mg/ml. The effective amount of the any of the compositions can be 16-19 mg/ml. The effective amount of the any of the compositions can be 19-20 mg/ml. The effective amount of the any of the compositions can be 6.5 mg/ml. The effective amount of the any of the compositions can be 13 mg/ml.

The method of treatment can comprise administering an effective amount of any of the compositions of the disclosure to the subject in need thereof, wherein the effective amount of the any of the compositions can be a dose volume of 5-40 ml. The effective amount of the any of the compositions can be a dose volume of 5-10 ml. The effective amount of the any of the compositions can be a dose volume of 10-15 ml. The effective amount of the any of the compositions can be a dose volume of 15-20 ml. The effective amount of the any of the compositions can be a dose volume of 20-25 ml. The effective amount of the any of the compositions can be a dose volume of 25-30 ml. The effective amount of the any of the compositions can be a dose volume of 30-35 ml. The effective amount of the any of the compositions can be a dose volume of 35-40 ml. The effective amount of the any of the compositions can be a dose volume of 10-20 ml. The effective amount of the any of the compositions can be a dose volume of 20-30 ml. The effective amount of the any of the compositions can be a dose volume of 30-40 ml. The effective amount of the any of the compositions can be a dose volume of 5 ml. The effective amount of the any of the compositions can be a dose volume of 10 ml. The effective amount of the any of the compositions can be a dose volume of 20 ml. The effective amount of the any of the compositions can be a dose volume of 30 ml. The effective amount of the any of the compositions can be a dose volume of 40 ml. The effective amount of the any of the compositions can be 6.5 mg/ml in a dose volume of 10 ml. The effective amount of the any of the compositions can be 13 mg/ml in a dose volume of 20 ml.

The present disclosure provides a method of treatment for post-surgical pain in a subject in need thereof, wherein the subject can be a mammal. The present disclosure provides a method of treatment for pain in a subject in need thereof, wherein the subject can be a mammal. The subject can be a human, a murine, an equine, a canine, a feline, a rabbit, a pig, a hamster, a bovine or a camelid. The subject can be a human. The human subject can be a male. The human subject can be a female.

The effective amount of the any of the compositions can be administered perineurally, epidurally, subcutaneously, intradermally, orally, intramuscularly or intravenously. The effective amount of the formulation can be administered perineurally. The effective amount of the formulation is administered by direct instillation into a wound near the site of surgical procedure. The effective amount of the formulation is administered by infiltration at the site of surgical procedure.

An ultrasound probe can be used for administering the effective amount of the any of the compositions to a site near a nerve distal to the site of surgery or injury of the subject in need thereof, wherein the targeted nerve provides innervation to the site of surgery or injury, with the aid of an ultrasound guided probe.

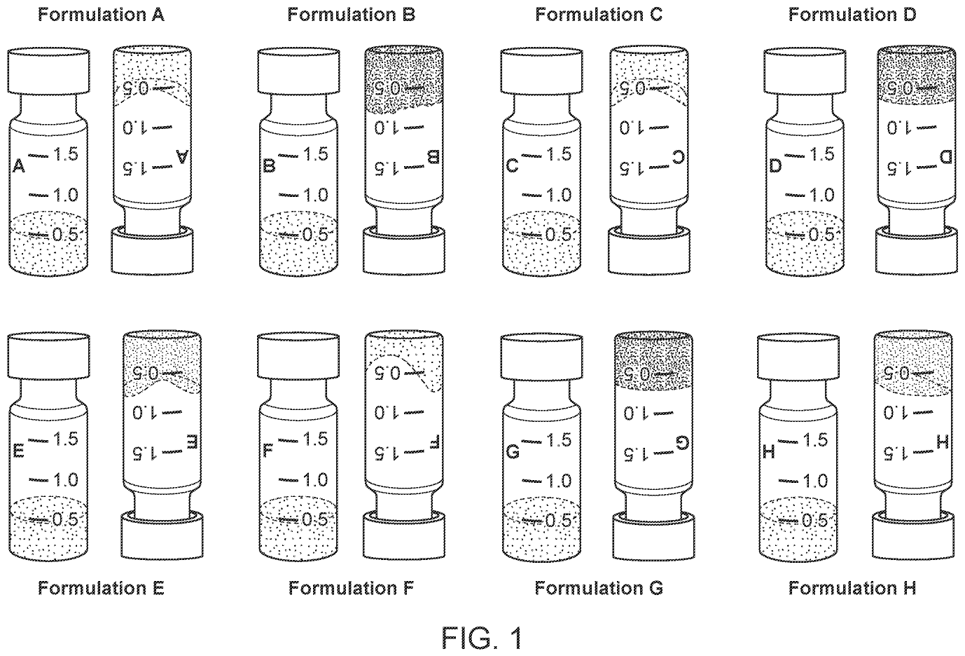

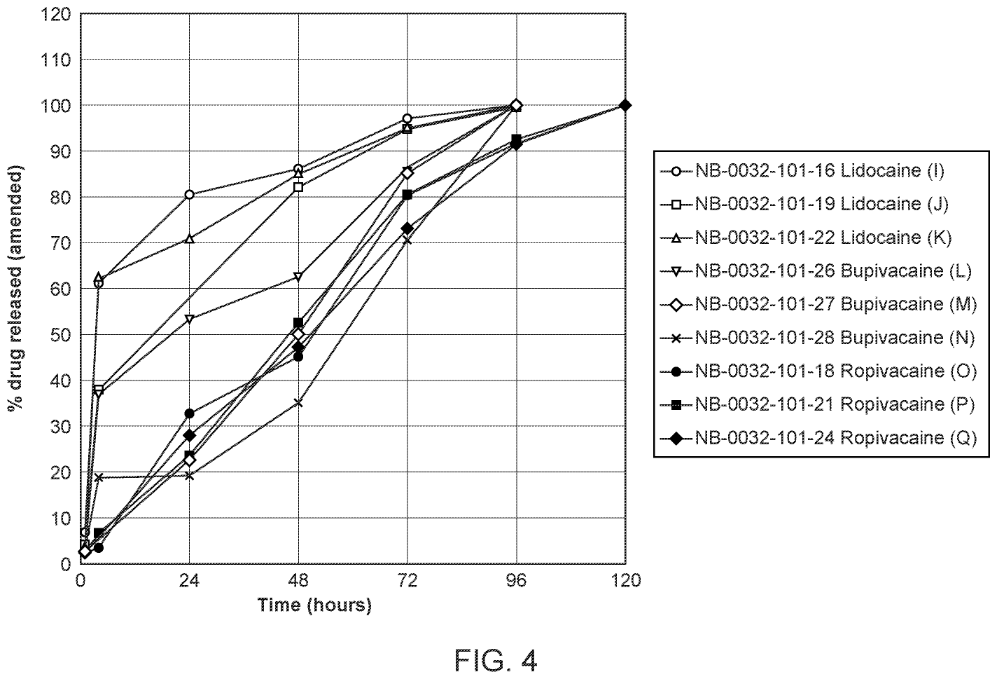

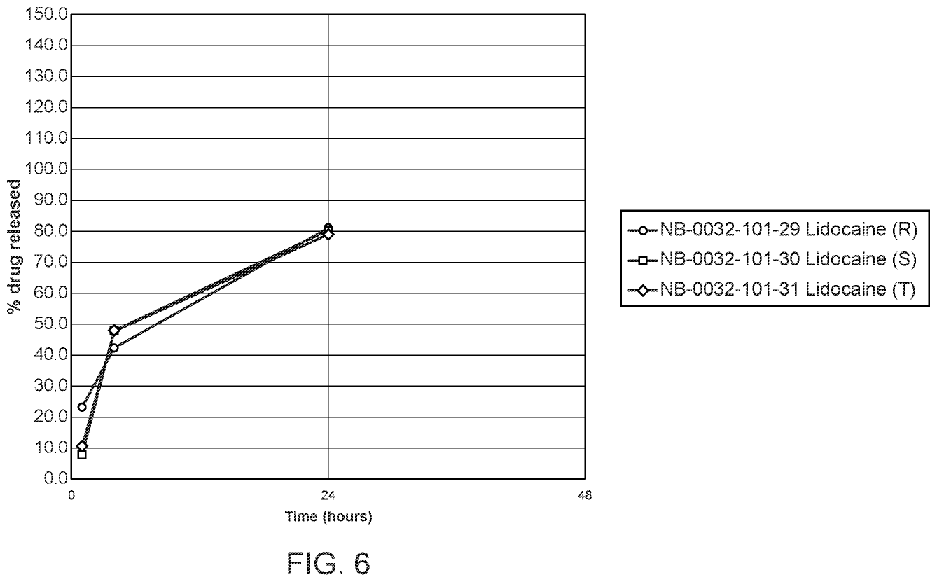

The results provided in the instant disclosure demonstrate steady and extended release kinetics of bupivacaine and ropivacaine (FIG. 4) and lidocaine (FIG. 6) from the composition disclosed herein. The results provided in the instant disclosure demonstrate that compositions of the disclosure are formulations of anesthetic drugs of amide group like lidocaine, bupivacaine and ropivacaine, that are have the optimal physical properties (CGT, Onset Temp, and MaxG′) that enable the formulations to be in liquid form at low temperature but undergo hydrogel formation upon increase in temperature (FIGS. 1, 3 and 5).

These results disclosed herein support the use of the compositions disclosed herein, is suitable for encapsulating the delivery of anesthetic drugs at the site of injection and can release the drug locally for several days. These results disclosed herein support the use of the compositions disclosed herein, in methods of treatment for, and management of post-surgical pain in a subject in need thereof while not materially impairing motor function.

The present disclosure provides a method of manufacturing any of the compositions disclosed herein. The method of manufacturing any of the compositions disclosed herein comprises: i) combining and dissolving: a) an amount of the PLGA-PEG-PLGA triblock polymer; b) an amount of water; c) an amount of the anesthetic drug; d) an amount of the polar organic solvent; and e) an amount of the solubility enhancer, to form a mixture; ii) stirring the mixture of (i) at 1-30° C.; iii) filtering the clear solution of (ii) through a sterile filter; and iv) collecting and freezing the filtered solution of (iii) at ≤−20° C.

In the method of manufacturing any of the compositions disclosed herein, the dissolved anesthetic drug solution is then combined with the polymer solution to stir at 8-12° C.

The method of manufacturing any of the compositions disclosed herein, can comprise in step i): 1) compounding and dissolving the amount of the PLGA-PEG-PLGA triblock polymer of (a) in the amount of water of (b) at 1-30° C., to form a polymer solution; 2) dissolving the amount of the anesthetic drug of (c) in the amount of the polar organic solvent of (d) and the amount of the solubility enhancer of (e), in a separate vessel to form an anesthetic drug solution; and 3) combining the dissolved anesthetic drug solution of (2) with the polymer solution of (1) to form a mixture. In the method of manufacturing any of the compositions disclosed herein, temperature of stirring the mixture can be 8-12° C.

In the method of manufacturing any of the compositions disclosed herein, the compounding and dissolving of the anesthetic drug, the PLGA-PEG-PLGA triblock polymer, the polar organic solvent, the solubility enhancer and water is done under controlled environmental conditions compatible with incorporation into an aseptic fill-finish process. In the method of manufacturing any of the compositions disclosed herein, the sterile filter is a 0.5/0.2 m sterile filter.

The present disclosure is further illustrated by the following examples that should not be construed as limiting. The contents of all references, patents, and published patent applications cited throughout this application, as well as the Figures, are incorporated herein by reference in their entirety for all purposes. The description will be further illustrated in the examples following below.

### EXAMPLES

### Example 1: Overview of Development of Hydrogel Formulations of Anesthetic Drugs

Bupivacaine, lidocaine, and ropivacaine are a few of the common anesthetics used in surgery for post-surgical pain management. Provided herein is a study describing the development of a thermosensitive hydrogel technology for the delivery of these active pharmaceutical ingredients (APIs) comprising anesthetics for the purpose of post-surgical pain management. The study described herein used lidocaine, bupivacaine, and ropivacaine as model actives; however, the formulations described herein are suitable for encapsulating the delivery of other lipophilic anesthetic and non-anesthetic APIs as well. The hydrogel was primarily composed of PLGA-PEG-PLGA triblock copolymer. When dissolved in aqueous media, the triblock polymers used in the study described herein formed micelles that allow the polymer to exist in a solution phase at ambient or refrigerated temperatures but forms a soft gel at body temperature. The temperature at which the formulations described herein transitioned from solution state to a gel state is referenced as the critical gelation temperature (CGT). As the formulations described herein were exposed to temperature above the CGT, the hydrophobic interactions between the inner domains of the micelles began to dominate, resulting in a gel network that is fully reversible upon cooling. The present study describes the development of formulations for the delivery of local anesthetics, wherein the formulations were designed to have a CGT just below the body temperature, allowing easy administration of the formulation. Once administered into the body, the liquid formulation described herein would quickly transition into a soft, pliable depot that remains localized at the site of injection and releases the drug locally for several days.

The formulations of the disclosure were developed from different PLGA-PEG-PLGA triblock polymers with varying block lengths ranging from 1100-1000-1100 Da to 1700-1500-1700 Da and varying lactide:glycolide (L:G) ratios from 100:0 (pure PLA) to 50:50 L:G PLGA. The polymers used for the development of the formulations of the disclosure, were tested at different concentrations but also mixed in various combinations, tailoring the rheological profile to better match the desired gelation properties, as described above. These polymers used in the study described herein are representative of a few different parameters that can be adjusted but still successfully used in a thermosensitive hydrogel. Table 1 summarizes the properties and identifiers of the three such polymers tested in the study described herein.

TABLE 1
Polymers tested in development of formulation

| | Target polymer | | Target polymer | |
|---|---|---|---|---|
| | molecular weight | | block molecular | PLGA L:G |
| Identifier | (Da) | Composition | weight (Da) | Ratio |
| P1 | 4300 | PLGA-PEG-PLGA | 1400-1500-1400 | 50:50 |
| P2 | 3200 | PLGA-PEG-PLGA | 1100-1000-1100 | 75:25 |
| P3 | 4900 | P(DL)LA-PEG-P(DL)LA | 1700-1500-1700 | 100:0 |

Because the bupivacaine, lidocaine, and ropivacaine (APIs or actives) are lipophilic molecules, the study described herein employed a solvent system to dissolve the APIs before combining with the aqueous polymeric solution. The results described herein show that although the polymeric micelles help with the solubilization of the APIs, pre-dissolving the APIs in a solvent allowed for quicker incorporation into the polymeric micelles and improved stability of the formulation. In order to determine a suitable solvent, the solubility of all three APIs were determined in dimethyl sulfoxide, ethyl acetate, methylene chloride (DCM), benzyl alcohol, and dimethylacetamide (DMA). The results described herein showed that DMA was the best common solvent for all three compounds and could achieve concentrations of 500 mg/mL, 200 mg/mL, and 80 mg/mL for lidocaine, bupivacaine, and ropivacaine respectively. The results described herein showed that DCM also solubilized bupivacaine but did not maintain adequate drug solubility when mixed with the polymer system to form the final formulation.

The development of the initial formulations of the study described herein focused on using the triblock copolymer P1 of Table 1, as a matrix to achieve the desired critical gelation temperature. The first formulation targeted a drug load of 10 mg/mL lidocaine in 2% DMA, 10% Polyethylene Glycol 400 (PEG 400), and 20% P1 (Table 2). PEG 400 was used as a co-solvent to further enhance solubility of the active in aqueous media and to decrease the gelation temperature to be close to but below body temperature. Rheological properties including critical gelation temperature (CGT), onset temperature, and elastic (G′) and viscous (G″) moduli were used in conjunction with visual and IVR experiments to analyze the impact of different formulation component compositions and concentrations on the suitability of the formulation in an extended-release postsurgical pain management indication.

### Example 2: Effect of PEG and Triblock Copolymer Concentrations on Solution to Gel Transition

Objective: The study described herein describes performing rheological analysis of the formulations disclosed herein, to evaluate the hypothesis that by increasing the PEG 400 and/or the polymer concentration, the number of micelles that can form a gel network would increase and therefore allow for a critical gelation point where the material transitions into an elastic gel rather than a liquid. This increase in polymer and PEG 400 concentrations would have consequential decreases in onset temperature. The onset temperature refers to the temperature at which the viscous moduli begins to increase in rheometry. The effect of the concentrations of PEG 400 and/or the polymer concentration on the measured critical gelation and onset temperatures of the formulations tested with P1 is summarized in Table 2.

TABLE 2
Formulations varying PEG400 and polymer concentration. did
not readily lead to a critical gelation temperature.

| | | | | | | | Onset | Max. |
|---|---|---|---|---|---|---|---|---|
| | Sample | | Target | | | CGT | temp. | G′ |
| ID | Name | Compound | conc. | | Solution | (degC.) | (degC.) | (Pa) |
| | NB-0032- | Lidocaine | 10 | mg/mL | 2% DMA + 10% PEG | N/A | 26 | 64 |
| | 101-01 | | | | 400 + 20% P1 | | | |
| A | NB-0032- | | | | 2% DMA + 10% PEG | N/A | 26 | 96 |
| | 101-03 | | | | 400 + 20% P1 | | | |
| B | NB-0032- | | | | 2% DMA + 15% PEG | 29 | 20 | 184 |
| | 101-04 | | | | 400 + 20% P1 | | | |
| C | NB-0032- | | | | 2% DMA + 11% PEG | N/A | 19 | 81 |
| | 101-09 | | | | 400 + 25% P1 | | | |
| D | NB-0032- | Bupivacaine | 4 | mg/mL | 10% DMA + 15% PEG | N/A | 12 | 85 |
| | 101-07 | | | | 400 + 23% P1 | | | |
| E | NB-0032- | | 3 | mg/mL | 5% DMA + 12% PEG | N/A | 17 | 63 |
| | 101-10 | | | | 400 + 25% P1 | | | |
| F | NB-0032- | | 10 | mg/mL | 5% DMA + 10% PEG | N/A | 21 | 20 |
| | 101-12 | | | | 400 + 25% P1 | | | |
| G | NB-0032- | Ropivacaine | 4 | mg/mL | 10% DMA + 15% PEG | N/A | 15 | 56 |
| | 101-08 | | | | 400 + 23% P1 | | | |
| H | NB-0032- | | 3 | mg/mL | 5% DMA + 12% PEG | N/A | 17 | 64 |
| | 101-11 | | | | 400 + 25% P1 | | | |

Observation: One formulation containing lidocaine demonstrated a noted increase in max G′ (related to stiffness of the formed depot) and CGT; however, for the formulations containing bupivacaine and ropivacaine, the increase in concentration of PEG 400 did not appear to have a significant impact on the overall gelation behavior. An increase in the P1 concentration in all the formulations similarly decreased the onset temperatures but with no significant impact on max G′. The visual sol-gel transition was tested by heating an aliquot of each formulation in a vial and placing in an incubator at 40° C. Once the formulations became fully opaque or after 20 minutes, the samples were removed, and the vials were inverted. If the formulations gelled, the depot demonstrated no movement (as noted for Formulations B, D, and G). However, if the formulation only transitioned into a viscous liquid, the liquid was observed to slowly flow downwards with gravity (as noted for Formulations A, C, E, F, and H). When heated, there was a visual change in almost all formulations of increased viscosity and increased opacity. (FIG. 1). The results described herein showed that addition of PEG 400 by 5% led to a 6° C. decrease in the onset temperature in some cases. The results described herein showed that only formulations B, D, G, and H stayed as a visible depot after media addition for IVR.

Conclusion: The results described herein showed that formulations B, D, and G had a higher amount of PEG, and formulations D and G did not have a defined CGT, suggesting that even without a clearly defined CGT, the addition of PEG increased the elastic moduli enough to form soft gel-like depots capable of maintaining structure and extending residency.

### Example 3: Invitro Release Study of Lidocaine, Bupivacaine and Ropivacaine from Formulations that Formed Visible Opaque Depot

Objective: Described herein is an in vitro release study using the formulations that stayed as a visible depot after media addition for IVR. The formulations that transitioned only to a viscous liquid (and not an opaque depot) also dissolved with the addition of media in in vitro release (IVR) studies. Such formulations were not further characterized for IVR profile. IVR was conducted by placing an aliquot of formulation into a 50 mL conical tube placed in a 37° C. water bath on a shaker. The formulation was left to gel before surfactant-containing media was slowly added as not to disturb any formed depot.

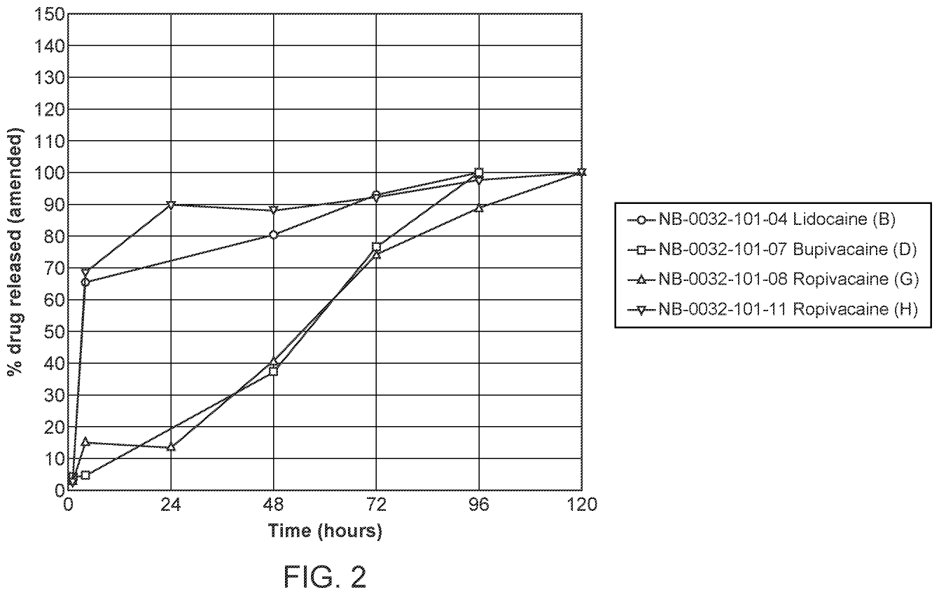

Observation: The results described herein showed measured cumulative drug release profile of the four formulations (B, D, G, and H) (FIG. 2). Samples B and H demonstrated a burst release, paying out most of their payload within the first four hours. The study concluded that lidocaine in sample B is less lipophilic than bupivacaine and ropivacaine and therefore could have a large driving force to escape the polymer matrix into the in vitro IVR media. The results described herein showed that sample H did not form an opaque gel in the visual sol-gel heating studies (FIG. 1) and thus likely was not forming a dense depot that would prolong the release of ropivacaine in sink conditions. The results described herein showed that samples D and G showed promising in vitro release profiles with only 50 and 70% drug load released after 72 hours, release rates much slower than the other formulations.

Conclusion: The results described herein showed that while the concentration of PEG and/or polymer have an effect on gelling properties of the formulations, the in vitro release rate of these formulations may be dependent on the properties of the drug.

### Example 4: Effect of Drug on Gelation Properties

Objective: The study described herein evaluated the effect of drug concentrations on rheological properties of the formulations.

Observation: Although expected to aid in gelation given their lipophilic nature, lidocaine at 10 mg/mL did not have a significant impact on gelation properties of the formulation. Formulations containing bupivacaine up to 3 mg/mL showed slight impact on the onset temperatures, leading to a quicker decline in moduli and loss of elastic and viscous properties at lower temperatures compared to the vehicle. This impact was increased as the bupivacaine drug load was increased, suggesting unexpected impedance in gelation from bupivacaine. Ropivacaine showed similar impact on the rheological properties as bupivacaine, demonstrating slight decrease on the onset temperatures and max G′ values when compared to the vehicle. Conclusion: The impact of the APIs on the rheological property of the formulation was not predictable and was therefore unanticipated.

### Example 5: Effect of Polymer Composition

Objective: The study described herein determined the effect of the polymer compositions on the rheological properties, solution to gel transition and IVR of the gel formulations described herein. In order to overcome this impact of the API, the study described herein evaluated other polymer compositions to achieve a better gel near body temperature. Due to poor solubility of the bupivacaine in some of the other polymer systems, the study described herein used, targeted a drug load of 5 mg/mL for these formulations.

Observation: The new polymer compositions resulted in lidocaine formulations with a distinct CGT and high max G′ values that were amenable to an injectable thermosensitive gel. The composition and rheological properties of the new polymer compositions is depicted in Table 3.

TABLE 3
Formulations varying polymer composition to achieve CGT

| | | | | | | | Onset | Max. |
|---|---|---|---|---|---|---|---|---|
| | Sample | | Target | | | CGT | temp. | G′ |
| ID | Name | Compound | conc. | | Solution | (degC.) | (degC.) | (Pa) |
| I | NB-0032- | Lidocaine | 10 | mg/mL | 2% DMA + 15% | 28.5 | 20 | 268 |
| | 101-16 | | | | PEG 400 + 17% P3 | | | |
| J | NB-0032- | | | | 2% DMA + 10% | 33.4 | 20 | 213 |
| | 101-19 | | | | PEG 400 + 24% | | | |
| | | | | | (78:22 P1:P2) | | | |
| K | NB-0032- | | | | 2% DMA + 15% | 32.5 | 22 | 197 |
| | 101-22 | | | | PEG 400 + 20% | | | |
| | | | | | (60:40 P1:P3) | | | |
| L | NB-0032- | Bupivacaine | 5 | mg/mL | 5% DMA + 15% | 29.3 | 20 | 193 |
| | 101-26 | | | | PEG 400 + 17% P3 | | | |
| M | NB-0032- | | | | 5% DMA + 10% | N/A | 18 | 63 |
| | 101-27 | | | | PEG 400 + 24% | | | |
| | | | | | (78:22 P1:P2) | | | |
| N | NB-0032- | | | | 5% DMA + 15% | N/A | 19 | 125 |
| | 101-28 | | | | PEG 400 + 20% | | | |
| | | | | | (60:40 P1:P3) | | | |
| O | NB-0032- | Ropivacaine | 4 | mg/mL | 5% DMA + 15% | 28.6 | 21 | 232 |
| | 101-18 | | | | PEG 400 + 17% P3 | | | |
| P | NB-0032- | | | | 5% DMA + 10% | N/A | 18 | 91 |
| | 101-21 | | | | PEG 400 + 24% | | | |
| | | | | | (78:22 P1:P2) | | | |
| Q | NB-0032- | | | | 5% DMA + 15% | 30.9 | 19 | 154 |
| | 101-24 | | | | PEG 400 + 20% | | | |
| | | | | | (60:40 P1:P3) | | | |

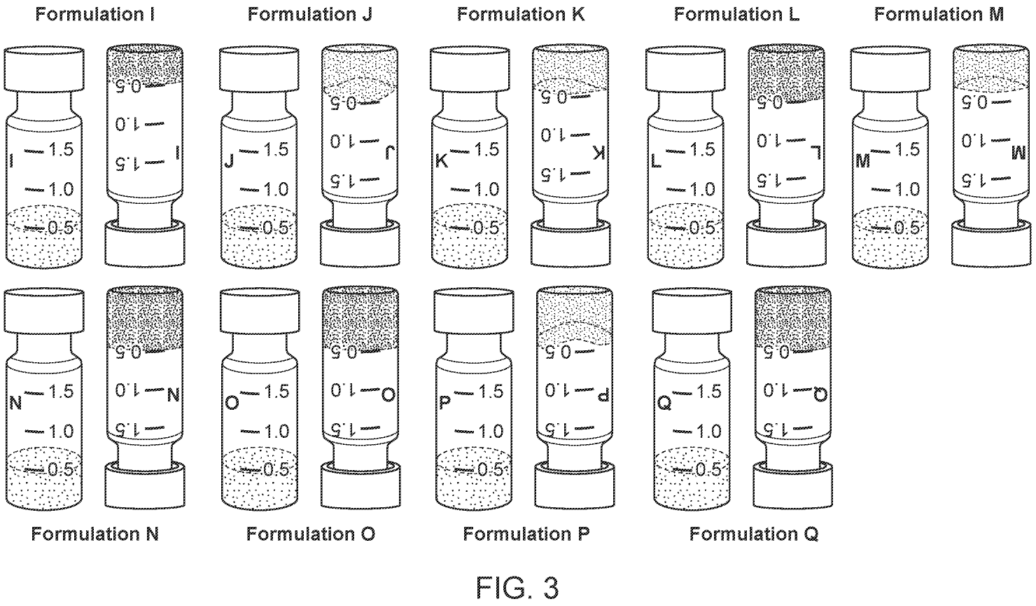

The results described herein showed that when incubated at 40° C. for visual observation of the sol-gel transition as described previously, formulations I, L, and O formed a gel depot that retained its shape and did not move when the vial was inverted (FIG. 3). When tested for IVR, all samples, in addition to the lead samples I, L, and O, showed a solid depot formation that remained intact upon media addition. The results of the IVR data analysis described herein showed lidocaine formulations (I, J, and K) were still releasing relatively quickly with only small improvements from sample B. However, formulations of both bupivacaine and ropivacaine had extended release over 72 hours from the formulations made with different polymers, even in cases without a defined CGT (FIG. 4).

Conclusion: Based on rheological properties of CGT, onset temperature, and maximum G′ values, a vehicle using P3 2-5% DMA and 15% PEG400 (samples I, L, and O) appeared to be the most promising for extended delivery of lidocaine, bupivacaine, and ropivacaine. The formulation for lidocaine needed further evaluation. The study described herein concluded that the burst behavior seen in lidocaine formulations may be due to the physicochemical properties of lidocaine itself, with a log P value of 2.4, compared to 3.4 and 2.9 of bupivacaine and ropivacaine, respectively.

### Example 6: Formulations for Extended Release Profile of Lidocaine

Objective: The study described herein determined the effect of additional formulations with the optimum rheological and solution to gel transition properties, to further extend the release profile of lidocaine. The additional formulations of lidocaine described herein were made with decreased PEG 400 and decreased DMA to further adjust the microenvironment surrounding the drug in the gel matrix. The objective of decreasing DMA in the study described herein was to decrease any small solubilizing benefit of DMA in the formulation in order to maximize the solubility enhancement from the polymer micelles. The objective of decreasing PEG 400 in the study described was to decrease the hydration of the gel and create a more hydrophobic matrix that impedes the diffusion rate of drug out of the system.

Observation: The formulation details and corresponding rheology data are shown in Table 4.

Table 4 shows the formulation details and corresponding rheology data.

| | | | | | | Onset | Max. |
|---|---|---|---|---|---|---|---|
| | Sample | | Target | | CGT | temp. | G′ |
| ID | Name | Compound | conc. | Solution | (degC.) | (degC.) | (Pa) |
| R | NB-0032- | Lidocaine | 10 mg/mL | 1% DMA + 5% PEG | N/A | 21 | 213 |
| | 101-29 | | | 400 + 23.5% P3 | | | |
| S | NB-0032- | | | 1% DMA + 5% PEG | N/A | 19 | 230 |
| | 101-30 | | | 400 + 26% | | | |
| | | | | (54:46 P1:P2) | | | |
| T | NB-0032- | | | 1% DMA + 5% PEG | 28.8 | 18 | 414 |
| | 101-31 | | | 400 + 23.5% | | | |
| | | | | (50:50 P2:P3) | | | |

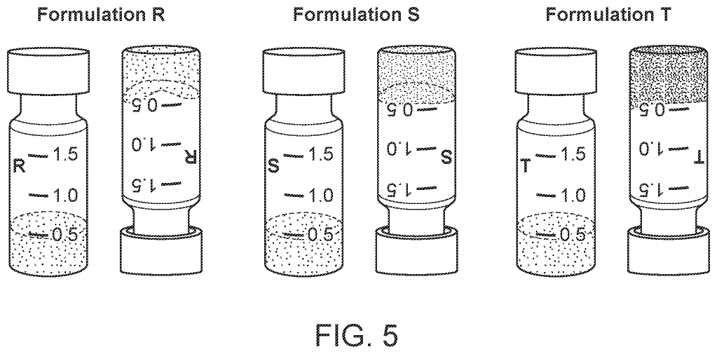

The results of visual sol-gel transition studies (FIG. 5) described herein, showed that sample T was the only sample that formed an opaque solid depot corresponding to the only one with a distinct CGT. The results of the IVR studies described herein showed that, all three samples gelled and showed a similar release profile (FIG. 6).

Conclusion: The results described herein showed that overall, having a lower PEG 400 content resulted decrease in the initial release rate of lidocaine as shown in these formulations and sample J, which had 10% PEG 400 when compared to those with 15% PEG (formulations B, I, and K) (FIG. 6).

The results of the study described herein showed that lidocaine, bupivacaine, and ropivacaine were all successfully encapsulated within a hydrogel system for extended release. The maximum drug encapsulation and release kinetics were impacted by the physicochemical properties of the API. Drug loads of 5 mg/mL bupivacaine and 3 mg/mL ropivacaine were encapsulated in multiple formulations made with 5-10 v/v % dimethyl acetamide (DMA), 10-15 v/v % polyethylene glycol 400 (PEG 400), 17-25 w/v % of polymers, and q.s. water, extending release for multiple days in vitro. The study described herein used multiple compositions of polylactic-co-glycolic acid-polyethylene glycol-polylactic-co-glycolic acid (PLGA-PEG-PLGA) with varying ratios of lactide:glycolide (L:G) and varying block lengths between 1000-1700 Da, as well as mixtures of polymer types to tailor the rheology profile.

The results described herein showed that lidocaine exhibited faster drug release than bupivacaine and ropivacaine, especially in the first 24 hours, however, lidocaine formulations still achieved 3-4 days of extended release in vitro. Multiple successful formulations were made with 1-2 v/v % DMA, 5-10 v/v % PEG 400, 17-25 w/v % of polymer, and q.s. water. PLA-PEG-PLA (1700-1500-1700 Da) was successfully used, as well as mixtures of PLGA-PEG-PLGA (1100-1000-1100 Da) with PLGA-PEG-PLGA (1400-1500-1400 Da) or the PLA-PEG-PLA. The results of the studies described herein show generation of novel compositions of drugs based on PLGA-PEG-PLGA triblock copolymer formulations, wherein the formulations depicted the solution to gel transition properties and in vitro drug release rate, suitable for use as a hydrogel formulation in delivering active pharmaceutical ingredients (APIs) for the purpose of post-surgical pain management.

### Example 7: FX301 Drug Product (DP) Manufacturing Process

Described herein is a process for manufacturing the composition of an anesthetic drug formulated in a triblock polymer of PLGA-PEG-PLGA, of the instant disclosure. FX301 DP is a sterile injectable solution comprised of 0.5% to 1.5% (w/v) anesthetic drug formulated in 19-25% (w/w) PLGA-PEG-PLGA (1500-1500-1500 Da, LA:GA 50:50), 1-2% (w/w) DMA, 10-11% (w/w) PEG 400, and water. The DMA and PEG 400 are used as drug solubilizers that dissolve the DS, anesthetic drug, from its crystalline form. The PLGA-PEG-PLGA is a functional excipient that confers extended release and physical duration at the site of injection vis-a-vis micellar encapsulation of the drug substance and formation of a thermo-sensitive hydrogel. This allows for injection of a low-viscosity solution, similar to that of current peripheral nerve block administration procedures which then quickly transitions into a gel depot upon injection into the body.

What is claimed is:

1. A composition of an anesthetic drug of amino amide group formulated in a triblock copolymer component, wherein the composition comprises:
a) the anesthetic drug at a concentration of 0.2 to 2% w/v;
b) the triblock copolymer component comprises one or more PLGA-PEG-PLGA triblock copolymers at a concentration of 5% to 35% w/v:
(i) wherein each Poly-Lactic-co-Glycolic acid (PLGA) polymer block has a molecular weight (MW) of 400 to 2550 Da,
(ii) wherein the Polyethylene Glycol (PEG) polymer block has a MW of 1000-3000 Da,
(iii) wherein the PLGA comprises 40% to 100% of lactic acid (LA) and 60% to 0% of glycolic acid (GA);
c) a polar organic solvent at a concentration of 0% to 20% v/v; and
d) a solubility enhancer at a concentration of 0% to 20% v/v,
and wherein any two PLGA-PEG-PLGA triblock copolymers are non-identical in total MW.

2. The composition of claim 1, wherein each PLGA polymer block has a MW of 1000 Da to 1500 Da.

3. The composition of claim 1, wherein each PLGA polymer block has a MW of 1500-2000 Da.

4. The composition of claim 1, wherein the PLGA comprises any one of:
a) 50% of lactic acid (LA) and 50% of glycolic acid (GA) (LA:GA ratio of 50:50);
b) 75% of lactic acid (LA) and 25% of glycolic acid (GA) (LA:GA ratio of 75:25);
c) 100% of lactic acid (LA) and 0% of glycolic acid (GA) (LA:GA ratio of 100:0).

5. The composition of claim 1, wherein the triblock copolymer component comprises a first PLGA-PEG-PLGA triblock copolymer and a second PLA-PEG-PLA triblock copolymer, wherein the first PLGA-PEG-PLGA triblock copolymer and the second PLGA-PEG-PLGA triblock copolymer comprise any one of:
a) a PLGA with 50% of lactic acid (LA) and 50% of glycolic acid (GA) (LA:GA ratio of 50:50), and a PLGA with 75% of lactic acid (LA) and 25% of glycolic acid (GA) (LA:GA ratio of 75:25), respectively;
b) a PLGA with 50% of lactic acid (LA) and 50% of glycolic acid (GA) (LA:GA ratio of 50:50), and a PLGA with 100% of lactic acid (LA) and 0% of glycolic acid (GA) (LA:GA ratio of 100:0), respectively; and
c) a PLGA with 75% of lactic acid (LA) and 25% of glycolic acid (GA) (LA:GA ratio of 75:25), and a PLGA with 100% of lactic acid (LA) and 0% of glycolic acid (GA) (LA:GA ratio of 100:0), respectively.

6. The composition of claim 5, wherein the triblock copolymer component comprises any one of:
a) a first PLGA-PEG-PLGA triblock copolymer with a PLGA polymer block of MW of 1200-1500 Da and a PEG polymer block of MW of 1500-2000 Da, and a second PLGA-PEG-PLGA triblock copolymer with a PLGA polymer block of MW of 1000-1200 Da and a PEG polymer block of MW of 1000-1500 Da;
b) a first PLGA-PEG-PLGA triblock copolymer with a PLGA polymer block of MW of 1200-1500 Da and a PEG polymer block of MW of 1500-2000 Da, and a second PLGA-PEG-PLGA triblock copolymer with a PLGA polymer block of MW of 1500-2000 Da and a PEG polymer block of MW of 1000-1500 Da; and
c) a first PLGA-PEG-PLGA triblock copolymer has a PLGA polymer block of MW of 1000-1200 Da and a PEG polymer block of MW of 1000-1500 Da, and a second PLGA-PEG-PLGA triblock copolymer has a PLGA polymer block of MW of 1500-2000 Da and a PEG polymer block of MW of 1000-1500 Da.

7. The composition of claim 6, wherein the triblock copolymer component comprises any one of:
a) a first PLGA-PEG-PLGA triblock copolymer having 1400 Da-1500 Da-1400 Da of PLGA-PEG-PLGA, and the second PLGA-PEG-PLGA triblock copolymer having 1100 Da-1000 Da-1100 Da of PLGA-PEG-PLGA;
b) a first PLGA-PEG-PLGA triblock copolymer having 1400 Da-1500 Da-1400 Da of PLGA-PEG-PLGA, and the second PLGA-PEG-PLGA triblock copolymer having 1700 Da-1500 Da-1700 Da of PLGA-PEG-PLGA; and
c) a first PLGA-PEG-PLGA triblock copolymer having 1100 Da-1000 Da-1100 Da of PLGA-PEG-PLGA, and the second PLGA-PEG-PLGA triblock copolymer having 1700 Da-1500 Da-1700 Da of PLGA-PEG-PLGA.

8. The composition of claim 7, wherein the triblock copolymer component comprises the first PLGA-PEG-PLGA triblock copolymer at a concentration of 50%-80% of the triblock copolymer component, and the second PLGA-PEG-PLGA triblock copolymer at a concentration of 20-50% of the triblock copolymer component.

9. The composition of claim 1, wherein the triblock copolymer component comprise one PLGA-PEG-PLGA triblock copolymer, wherein the PLGA-PEG-PLGA triblock copolymer comprises any one of:
a) 50% of lactic acid (LA) and 50% of glycolic acid (GA) (LA:GA ratio of 50:50);
b) 75% of lactic acid (LA) and 25% of glycolic acid (GA) (LA:GA ratio of 75:25); and
c) 100% of lactic acid (LA) and 0% of glycolic acid (GA) (LA:GA ratio of 100:0).

10. The composition of claim 9, wherein the PLGA-PEG-PLGA triblock copolymer has any one of,
a) a PLGA polymer block of MW of 1200-1500 Da and a PEG polymer block of MW of 1500-2000 Da;
b) a PLGA polymer block of MW of 1000-1200 Da and a PEG polymer block of MW of 1000-1500 Da; and
c) a PLA polymer block of MW of 1500-2000 Da and a PEG polymer block of MW of 1000-1500 Da.

11. The composition of claim 10, wherein the PLGA-PEG-PLGA triblock copolymer is any one of:
a) a 1400 Da-1500 Da-1400 Da of PLGA-PEG-PLGA;
b) 1100 Da-1000 Da-1100 Da of PLGA-PEG-PLGA; and
c) 1700 Da-1500 Da-1700 Da of PLGA-PEG-PLGA.

12. The composition of claim 1, wherein the anesthetic drug of amino amide group can be any one of a bupivacaine, lidocaine, and ropivacaine.

13. The composition of claim 12, wherein the anesthetic drug is any one of:
a) lidocaine at a concentration of 0.2% to 1% w/v;
b) bupivacaine at a concentration of 0.3% to 1% w/v; and
c) ropivacaine at a concentration of 0.3% to 0.4% w/v.

14. The composition of claim 1, wherein the polar organic solvent concentration is 1% to 20% v/v.

15. The composition of claim 14, wherein the polar organic solvent is Dimethyl acetamide (DMA), Dimethyl sulfoxide (DMSO), Glycofurol, N-Methyl-2-Pyrrolidone (NMP), or any combination thereof.

16. The composition of claim 1, wherein the solubility enhancer concentration is 5% to 20% v/v.

17. The composition of claim 16, wherein the solubility enhancer is Propylene Glycol (PG), Cremophor EL, Cremophor RH 60, Ethanol, Glycerin, PEG 300, PEG 400, polysorbates, Vitamin E-TPGS, PLGA-PEG diblock copolymers, hydroxypropyl-β-cyclodextrin, sulfobutyl ether-β-cyclodextrin, poloxamer 188, poloxomer 407, polyvinyl pyrrolidone (PVP), glycerol formal, Solutol HS, or any combination thereof.

18. The composition of claim 1, wherein the PLGA-PEG-PLGA triblock copolymer component concentration is 10% to 30% w/v.

19. The composition of claim 1, wherein the total molecular weight any of the one or more triblock copolymers of the triblock copolymer component is 2000-7000 Da.

20. The composition of claim 1, wherein the formulation comprises:
a) lidocaine at a concentration of 1% w/v;
b) a PLGA-PEG-PLGA triblock copolymer component at a concentration of 20% w/v:
(i) wherein the PLGA-PEG-PLGA triblock copolymer component comprises one 1400 Da-1500 Da-1400 Da of PLGA-PEG-PLGA triblock copolymer, and
(ii) wherein the PLGA comprises 50% of lactic acid (LA) and 50% of glycolic acid (GA) (LA:GA ratio of 50:50);
c) DMA at a concentration of 2% v/v; and
d) PEG 400 at a concentration of 15% v/v.

21. The composition of claim 1, wherein the formulation comprises:
a) lidocaine at a concentration of 1% w/v;
b) a PLGA-PEG-PLGA triblock copolymer component at a concentration of 17% w/v:
(i) wherein the PLGA-PEG-PLGA triblock copolymer component comprises one 1700 Da-1500 Da-1700 Da of PLGA-PEG-PLGA triblock copolymer, and
(ii) wherein the PLGA comprises 100% of lactic acid (LA) and 0% of glycolic acid (GA) (LA:GA ratio of 100:0);
c) DMA at a concentration of 2% v/v; and
d) PEG 400 at a concentration of 15% v/v.

22. The composition of claim 1, wherein the formulation comprises:
a) lidocaine at a concentration of 1% w/v;
b) a PLGA-PEG-PLGA triblock copolymer component at a concentration of 23.5% w/v:
(i) wherein the PLGA-PEG-PLGA triblock copolymer component comprises one 1700 Da-1500 Da-1700 Da of PLGA-PEG-PLGA triblock copolymer, and
(ii) wherein the PLGA comprises 100% of lactic acid (LA) and 0% of glycolic acid (GA) (LA:GA ratio of 100:0);
c) DMA at a concentration of 1% v/v; and
d) PEG 400 at a concentration of 5% v/v.

23. The composition of claim 1, wherein the formulation comprises:
a) lidocaine at a concentration of 1% w/v;
b) a PLGA-PEG-PLGA triblock copolymer component at a concentration of 26% w/v:
(i) wherein the PLGA-PEG-PLGA triblock copolymer component comprises a first and a second PLGA-PEG-PLGA triblock copolymers;
(ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA;
(iii) wherein the first PLGA-PEG-PLGA triblock copolymer comprises 50% of lactic acid (LA) and 50% of glycolic acid (GA) (LA:GA ratio of 50:50);
(iv) wherein the second PLGA-PEG-PLGA triblock copolymer comprises 75% of lactic acid (LA) and 25% of glycolic acid (GA) (LA:GA ratio of 75:25); and
(v) wherein the triblock copolymer component comprises the first PLGA-PEG-PLGA triblock copolymer at a concentration of 54% of the triblock copolymer component and the second PLGA-PEG-PLGA triblock copolymer at a concentration of 46% of the triblock copolymer component;
c) DMA at a concentration of 1% v/v; and
d) PEG 400 at a concentration of 5% v/v.

24. The composition of claim 1, wherein the formulation comprises:
a) lidocaine at a concentration of 1% w/v;
b) a PLGA-PEG-PLGA triblock copolymer component at a concentration of 23.5% w/v:
(i) wherein the PLGA-PEG-PLGA triblock copolymer component comprises a first and a second PLGA-PEG-PLGA triblock copolymers;
(ii) wherein the first PLGA-PEG-PLGA triblock copolymer is a 1100 Da-1000 Da-1100 Da triblock copolymer and the second PLGA-PEG-PLGA triblock copolymer is a 1700 Da-1500 Da-1700 Da of PLGA-PEG-PLGA;
(iii) wherein the first PLGA-PEG-PLGA triblock copolymer comprises 75% of lactic acid (LA) and 25% of glycolic acid (GA) (LA:GA ratio of 75:25);
(iv) wherein the second PLGA-PEG-PLGA triblock copolymer comprises 100% of lactic acid (LA) and 0% of glycolic acid (GA) (LA:GA ratio of 100:0); and
(v) wherein the triblock copolymer component comprises the first PLGA-PEG-PLGA triblock copolymer at a concentration of 50% of the triblock copolymer component and the second PLGA-PEG-PLGA triblock copolymer at a concentration of 40% of the triblock copolymer component;
c) DMA at a concentration of 1% v/v; and
d) PEG 400 at a concentration of 5% v/v.

25. The composition of claim 1, wherein the formulation comprises:
a) bupivacaine at a concentration of 1% w/v;
b) a PLGA-PEG-PLGA triblock copolymer component at a concentration of 23% w/v:
(i) wherein the PLGA-PEG-PLGA triblock copolymer component comprises one 1400 Da-1500 Da-1400 Da of PLGA-PEG-PLGA triblock copolymer, and
(ii) wherein the PLGA comprises 50% of lactic acid (LA) and 50% of glycolic acid (GA) (LA:GA ratio of 50:50);
c) DMA at a concentration of 10% v/v; and
d) PEG 400 at a concentration of 15% v/v.

26. The composition of claim 1, wherein the formulation comprises:
a) bupivacaine at a concentration of 0.5% w/v;
b) a PLGA-PEG-PLGA triblock copolymer component at a concentration of 17% w/v:
(i) wherein the PLGA-PEG-PLGA triblock copolymer component comprises one 1100 Da-1000 Da-1100 Da of PLGA-PEG-PLGA triblock copolymer, and
(ii) wherein the PLGA comprises 100% of lactic acid (LA) and 0% of glycolic acid (GA) (LA:GA ratio of 100:0);
c) DMA at a concentration of 5% v/v; and
d) PEG 400 at a concentration of 15% v/v.

27. The composition of claim 1, wherein the formulation comprises:
a) bupivacaine at a concentration of 0.5% w/v;
b) a PLGA-PEG-PLGA triblock copolymer component at a concentration of 24% w/v:
(i) wherein the PLGA-PEG-PLGA triblock copolymer component comprises a first and a second PLGA-PEG-PLGA triblock copolymers;
(ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA;
(iii) wherein the first PLGA-PEG-PLGA triblock copolymer comprises 50% of lactic acid (LA) and 50% of glycolic acid (GA) (LA:GA ratio of 50:50);
(iv) wherein the second PLGA-PEG-PLGA triblock copolymer comprises 75% of lactic acid (LA) and 25% of glycolic acid (GA) (LA:GA ratio of 75:25); and
(v) wherein the triblock copolymer component comprises the first PLGA-PEG-PLGA triblock copolymer at a concentration of 78% of the triblock copolymer component and the second PLGA-PEG-PLGA triblock copolymer at a concentration of 22% of the triblock copolymer component;
c) DMA at a concentration of 5% v/v; and
d) PEG 400 at a concentration of 10% v/v.

28. The composition of claim 1, wherein the formulation comprises:
a) bupivacaine at a concentration of 0.5% w/v;
b) a PLGA-PEG-PLGA triblock copolymer component at a concentration of 20% w/v:
(i) wherein the PLGA-PEG-PLGA triblock copolymer component comprises a first and a second PLGA-PEG-PLGA triblock copolymers;
(ii) wherein the first PLGA-PEG-PLGA triblock copolymer is a 1400 Da-1500 Da-1400 Da triblock copolymer and the second PLGA-PEG-PLGA triblock copolymer is a 1700 Da-1500 Da-1700 Da of PLGA-PEG-PLGA;
(iii) wherein the first PLGA-PEG-PLGA triblock copolymer comprises 50% of lactic acid (LA) and 50% of glycolic acid (GA) (LA:GA ratio of 50:50);
(iv) wherein the second PLGA-PEG-PLGA triblock copolymer comprises 100% of lactic acid (LA) and 0% of glycolic acid (GA) (LA:GA ratio of 100:0); and
(v) wherein the triblock copolymer component comprises the first PLGA-PEG-PLGA triblock copolymer at a concentration of 60% of the triblock copolymer component and the second PLGA-PEG-PLGA triblock copolymer at a concentration of 40% of the triblock copolymer component;
c) DMA at a concentration of 5% v/v; and
d) PEG 400 at a concentration of 15% v/v.

29. The composition of claim 1, wherein the formulation comprises:
a) ropivacaine at a concentration of 0.4% w/v;
b) a PLGA-PEG-PLGA triblock copolymer component at a concentration of 23% w/v:
(i) wherein the PLGA-PEG-PLGA triblock copolymer component comprises one 1400 Da-1500 Da-1400 Da of PLGA-PEG-PLGA triblock copolymer, and
(ii) wherein the PLGA comprises 50% of lactic acid (LA) and 50% of glycolic acid (GA) (LA:GA ratio of 50:50);
c) DMA at a concentration of 10% v/v; and
d) PEG 400 at a concentration of 15% v/v.

30. The composition of claim 1, wherein the formulation comprises:
a) ropivacaine at a concentration of 0.4% w/v;
b) a PLGA-PEG-PLGA triblock copolymer component at a concentration of 17% w/v:
(i) wherein the PLGA-PEG-PLGA triblock copolymer component comprises one 1100 Da-1000 Da-1100 Da of PLGA-PEG-PLGA triblock copolymer, and
(ii) wherein the PLGA comprises 100% of lactic acid (LA) and 0% of glycolic acid (GA) (LA:GA ratio of 100:0);
c) DMA at a concentration of 5% v/v; and
d) PEG 400 at a concentration of 15% v/v.

31. The composition of claim 1, wherein the formulation comprises:
a) ropivacaine at a concentration of 0.4% w/v;
b) a PLGA-PEG-PLGA triblock copolymer component at a concentration of 24% w/v:
(i) wherein the PLGA-PEG-PLGA triblock copolymer component comprises a first and a second PLGA-PEG-PLGA triblock copolymers;
(ii) wherein the first PLGA-PEG-PLGA triblock copolymer is a 1400 Da-1500 Da-1400 Da triblock copolymer and the second PLGA-PEG-PLGA triblock copolymer is a 1100 Da-1000 Da-1100 Da of PLGA-PEG-PLGA;
(iii) wherein the first PLGA-PEG-PLGA triblock copolymer comprises 50% of lactic acid (LA) and 50% of glycolic acid (GA) (LA:GA ratio of 50:50);
(iv) wherein the second PLGA-PEG-PLGA triblock copolymer comprises 75% of lactic acid (LA) and 25% of glycolic acid (GA) (LA:GA ratio of 75:25); and
(v) wherein the triblock copolymer component comprises the first PLGA-PEG-PLGA triblock copolymer at a concentration of 78% of the triblock copolymer component and the second PLGA-PEG-PLGA triblock copolymer at a concentration of 22% of the triblock copolymer component;
c) DMA at a concentration of 5% v/v; and
d) PEG 400 at a concentration of 10% v/v.

32. The composition of claim 1, wherein the formulation comprises:
a) ropivacaine at a concentration of 0.4% w/v;
b) a PLGA-PEG-PLGA triblock copolymer component at a concentration of 20% w/v:
(i) wherein the PLGA-PEG-PLGA triblock copolymer component comprises a first and a second PLGA-PEG-PLGA triblock copolymers;
(ii) wherein the first PLGA-PEG-PLGA triblock copolymer is a 1400 Da-1500 Da-1400 Da triblock copolymer and the second PLGA-PEG-PLGA triblock copolymer is a 1700 Da-1500 Da-1700 Da of PLGA-PEG-PLGA;
(iii) wherein the first PLGA-PEG-PLGA triblock copolymer comprises 50% of lactic acid (LA) and 50% of glycolic acid (GA) (LA:GA ratio of 50:50);
(iv) wherein the second PLGA-PEG-PLGA triblock copolymer comprises 100% of lactic acid (LA) and 0% of glycolic acid (GA) (LA:GA ratio of 100:0); and
(v) wherein the triblock copolymer component comprises the first PLGA-PEG-PLGA triblock copolymer at a concentration of 60% of the triblock copolymer component and the second PLGA-PEG-PLGA triblock copolymer at a concentration of 40% of the triblock copolymer component;
c) DMA at a concentration of 5% v/v; and
d) PEG 400 at a concentration of 15% v/v.

33. The composition of any one of claims 1-57, wherein the composition has a dose volume of 5 ml to 40 ml.

34. The composition of claim 1, for use as a medicament for prevention or treatment of pain in a subject in need thereof, wherein the medicament comprises an effective amount of the composition.

35. The composition for use according to claim 34, wherein the pain is any one of acute pain, pain during a surgical procedure or post-surgical pain.

36. The composition for use according to claim 34, wherein the effective amount induces an analgesic nerve block.

37. The composition for use according to claim 34, wherein the subject is a mammal.

38. A method of manufacturing a composition of claim 1, the method comprising:
i) combining and dissolving:
a) an amount of the one or more PLGA-PEG-PLGA triblock polymers;
b) an amount of water;
c) an amount of the anesthetic drug;
d) an amount of the polar organic solvent; and
e) an amount of the solubility enhancer;
to form a mixture;
ii) stirring the mixture of (i) at 1-30° C.;
iii) filtering the clear solution of (ii) through a sterile filter; and
iv) collecting and freezing the filtered solution of (iii) at ≤−20° C.

39. The method of claim 38, wherein step i) comprises:
1) compounding and dissolving the amount of the PLGA-PEG-PLGA triblock polymer of (a) in the amount of water of (b) at 1-30° C., to form a polymer solution;
2) dissolving the amount of the anesthetic drug of (c) in the amount of the polar organic solvent of (d) and the amount of the solubility enhancer of (e), in a separate vessel to form an anesthetic drug solution; and
3) combining the dissolved anesthetic drug solution of (1) with the polymer solution of (2) to form a mixture.

40. The method of claim 39, wherein the combining of (3) is done at a temperature of 8-12° C.
